(19) United States
(12) Patent Application Publication
(10) Pub. No.: US 2019/0345527 A1
(43) Pub. Date: Nov. 14, 2019

(54) METHOD FOR SYNTHESIZING DOUBLE-STRANDED DNA

(71) Applicant: NATIONAL UNIVERSITY CORPORATION KOBE UNIVERSITY, Kobe (JP)

(72) Inventors: Kenji TSUGE, Kobe (JP); Shunsuke TAKAHASHI, Kobe (JP); Akihiko KONDO, Kobe (JP)

(21) Appl. No.: 16/185,106

(22) Filed: Nov. 9, 2018

(30) Foreign Application Priority Data
May 14, 2018 (JP) 2018-093024

(51) Int. Cl. C12P 19/34; C12Q 1/6806; C12Q 1/686; C12Q 1/6876

(52) U.S. Cl. CPC C12P 19/34; C12Q 1/6876; C12Q 1/686; C12Q 1/6806

(57) ABSTRACT

The object of the present invention is to provide a method for synthesizing a double-stranded DNA fragment using a PCR method, the method being capable of accurately, easily, efficiently, and quickly synthesizing a double-stranded DNA fragment of interest, regardless of the sequence thereof.

The present invention is a method for synthesizing a double-stranded DNA by connecting short double-stranded DNAs to each other by overlap extension PCR to obtain a double-stranded DNA fragment of interest, the method having a plurality of stages of annealing temperature setting in a PCR cycle of the overlap extension PCR.

### BACKGROUND OF THE INVENTION

### (1) Field of the Invention

The present invention relates to a novel method for synthesizing a double-stranded DNA.

### (2) Description of the Related Art

In the field of genetic engineering, DNA synthesis for constructing a long chain DNA having a novel sequence has been performed for a variety of purposes. Methods for synthesizing the long chain DNA are largely composed of two steps. The first step is a step of synthesizing a double-stranded DNA fragment from a large number of single-stranded DNA oligonucleotides (oligo DNA). The second step is a step of constructing a long chain DNA by assembling a large number of double-stranded DNA fragments obtained in the first step. As the methods for constructing the long chain DNA, there are known methods such as a yeast assembly method (see Gibson, D. G, et al. Science, 5867, 1215-1220., 2008), Gibson assembly method (see Gibson, D. G, et al. Nat. Methods, 6, 343-345., 2009), Golden gate method (see Engler, C., et al. PLoS ONE, 4, e5553., 2009), LCR method (see Stefan de Kok, S., et al. ACS Synth. Biol., 3, 97-106., 2014), and OGAB method (see Tsuge, K., et al. Nucleic Acids Res., 31, e133., 2003, Tsuge, K., et al. Sci. Rep., 5, 10655., 2015).

As the method for synthesizing the double-stranded DNA fragments described above, there are some known methods in which a large number of single-stranded oligo DNAs containing homologous regions are simultaneously bonded to each other for example in a PCR reaction solution and assembled to synthesize a double-stranded DNA fragment (see U.S. Patent Application Publication No. US20080182296 A1; Stemmer, W. P., et al. Gene, 164, 49-53., 1995; Xiong, A., et al. Nat. protoc., 1,791., 2006; Xiong, A. S., et al. Nucleic Acids Res., 32, e98-e98., 2004). In these methods, since a large number of single-stranded oligo DNAs are mixed in one PCR reaction solution, when these single-stranded oligo DNAs are bonded by PCR reaction, it leads to inconvenient situations such as the occurrence of mismatch between single-stranded oligo DNAs and the formation of the primer dimer. For these reasons, in these synthesis methods, it cannot be always synthesized a double-stranded DNA fragment for whatever DNA sequence to be synthesized is. In particular, when the DNA sequence of interest to be synthesized is a complex DNA sequence comprising a long repeat sequence, a sequence of contiguous identical nucleotides, or the like, the synthesis may be difficult or impossible.

Further, in these synthetic methods, in order to synthesize a double-stranded DNA having a DNA sequence of interest, it is necessary to design single-stranded oligo DNAs as the starting materials. When designing these single-stranded oligo DNAs, in order to set an annealing temperature for PCR reaction, it is necessary to adjust the length of the homologous regions for bonding a large number of single-stranded oligo DNAs. In addition, to prevent hairpin formation or the like of the single-stranded oligo DNAs in the synthesis step, it is necessary to adjust the length, the number, or the configuration of GC content or the like in each single-stranded oligo DNA. That is, in the previous synthesis method, the design of single-stranded oligo DNAs of the DNA sequence of interest to be synthesized is complicated, and the attempt to synthesize a large number of double-stranded DNA fragments of several to several-hundred units requires the design of single-stranded oligo DNAs for each of DNA sequences thereof, thus it occurs inconvenience that it takes time and effort to design the single-stranded oligo DNAs.

In the synthesis of the long chain DNA in the second step described above, a large number of double-stranded DNA fragments obtained in the first step are used as the assembly materials. When assembling the double-stranded DNA fragments in the synthesis of the long chain DNA in the second step, even the lack of one double-stranded DNA fragment makes it impossible to synthesize the long chain DNA of interest. However, in the previous synthesis methods of double-stranded DNA fragments, it occurs inconvenience that it takes time and effort until a large number of double-stranded DNA fragments of several to several hundred units are synthesized, and the synthesis is sometimes difficult or impossible depending on the complexity of the DNA sequence of interest to be synthesized, thus it has been difficult in the previous methods to quickly obtain all of the double-stranded DNA fragments to be used as the assembly materials. That is, in the previous synthesis methods of double-stranded DNA fragments, the double-stranded DNA fragments to be used as the assembly materials for synthesizing a long-chain DNA cannot be supplied quickly, which has been substantially a bottleneck. Thus, a new method capable of synthesizing double-stranded DNA fragments efficiently and quickly, which becomes the alternative to the previous synthesis methods, has been strongly desired.

Under these circumstances, the present inventors have conducted the study to solve the inconvenience described above and to develop a method that can accurately, easily, efficiently and quickly synthesize a double-stranded DNA fragment which can be used as an assembly material for long-chain DNA synthesis. That is, the object of the present invention is to provide a method for synthesizing a double-stranded DNA fragment using a PCR method, the method being capable of accurately, easily, efficiently, and quickly synthesizing a double-stranded DNA fragment of interest, regardless of the sequence thereof.

### BRIEF SUMMARY OF THE INVENTION

The present inventors have conducted intensive studies to solve the above problems, and found that, in the method for synthesizing a double-stranded DNA by connecting short double-stranded DNAs to each other by overlap extension PCR to obtain a double-stranded DNA fragment of interest, having a plurality of stages of annealing temperature setting in a PCR cycle allows to synthesize a double-stranded DNA fragment of interest accurately, easily, efficiently and quickly, and have completed the present invention. Namely, the gist of the present invention is as follows.

[1] A method for synthesizing a double-stranded DNA by connecting short double-stranded DNAs to each other by overlap extension PCR to obtain a double-stranded DNA fragment of interest, the method having a plurality of stages of annealing temperature setting in a PCR cycle of the overlap extension PCR.
[2] The method for synthesizing the double-stranded DNA according to [1], wherein the annealing temperature setting is performed at 2 to 20 stages.
[3] The method for synthesizing the double-stranded DNA according to [1] or [2], wherein an annealing temperature of the annealing temperature setting is 65 to 85° C.
[4] The method for synthesizing the double-stranded DNA according to any one of [1] to [3], wherein a temperature holding period in each stage of the annealing temperature setting is 10 seconds or more and 2 minutes or less.
[5] The method for synthesizing the double-stranded DNA according to any one of [1] to [4], wherein an overlapping region of DNA in the overlap extension PCR has 10 to 40 bases.
[6] The method for synthesizing the double-stranded DNA according to any one of [1] to [5], wherein 3 to 20 kinds of the short double-stranded DNAs are connected to each other in the overlap extension PCR.
[7] The method for synthesizing the double-stranded DNA according to any one of [1] to [6], wherein the short double-stranded DNAs used in the overlap extension PCR are synthesized by primer extension PCR.
[8] The method for synthesizing the double-stranded DNA according to [7], wherein the method has a plurality of stages of annealing temperature setting in a PCR cycle of the primer extension PCR.
[9] The method for synthesizing the double-stranded DNA according to [7] or [8], wherein a size of each of the short double-stranded DNAs synthesized by the primer extension PCR is 100 to 300 bases.
[10] A method for synthesizing a double-stranded DNA, the method comprising a primer extension PCR step of synthesizing short double-stranded DNAs by primer extension PCR, and an overlap extension PCR step of connecting the double-stranded DNAs synthesized in the primer extension PCR step to each other by overlap extension PCR to synthesize a double-stranded DNA fragment of interest, and the method having a plurality of stages of annealing temperature setting in a PCR cycle of the overlap extension PCR.

According to the present invention, by having a plurality of stages of annealing temperature setting in a PCR cycle, it becomes applicable to the bonding of a plurality of DNAs having different Tm values, and thus it can assemble a large number of single-stranded DNAs containing homologous regions easily, accurately, efficiently, and quickly. Further, since the double-stranded DNA synthesis method of the present invention is applicable to the bonding of DNAs having different Tm values, the method does not require labor to optimize the single-stranded oligo DNA sequences used as the materials or does not need to delicately reset the temperature for each double-stranded DNA fragment of interest. Furthermore, according to the present invention, a double-stranded DNA fragment of interest can be synthesized easily, accurately, efficiently, and quickly, regardless of the sequence thereof, so that even a double-stranded DNA fragment having a conventional complex sequence (for example, a long repeat sequence, a sequence of contiguous identical nucleotides, or AT-rich or GC-rich sequence), which has been hitherto difficult or impossible to synthesize, can be synthesized.

### BRIEF DESCRIPTION OF THE SEVERAL VIEWS OF THE DRAWING

FIG. 1 is a drawing schematically showing the double-stranded DNA synthesis method of the present invention;

FIG. 2 is a picture showing an investigation result of PCR conditions of the double-stranded DNA synthesis method of the present invention;

FIG. 3 is a picture showing an investigation result of PCR conditions of the double-stranded DNA synthesis method of the present invention;

FIG. 4 is pictures showing investigation results of PCR conditions of the double-stranded DNA synthesis method of the present invention;

FIG. 5 is a picture showing a result of comparison of the double-stranded DNA fragments obtained by the conventional synthesis method to that obtained by the synthesis method of the present invention;

FIG. 6 is a picture showing a result of electrophoresis of a plurality of double-stranded DNAs having different sequences which are simultaneously synthesized by the double-stranded DNA synthesis method of the present invention;

FIG. 7 is a diagram showing a result of comparison of the quality of the chemically synthesized single-stranded DNAs;

FIG. 8 is a picture showing a result of comparison of the double-stranded DNA fragments obtained by the conventional synthesis method to that obtained by the synthesis method of the present invention;

FIG. 9 is a picture showing a result of comparison of the double-stranded DNA fragments obtained by the conventional synthesis method to that obtained by the synthesis method of the present invention; and

FIG. 10 is a picture showing a result of comparison of the double-stranded DNA fragments obtained by the conventional synthesis method to that obtained by the synthesis method of the present invention.

### DETAILED DESCRIPTION OF THE INVENTION

Hereinafter, the double-stranded DNA synthesis method of the present invention will be described in detail. In this specification, unless otherwise stated, molecular biological techniques can be performed in accordance with the method described in the general experimental specification known to those skilled in the art or the methods conforming thereto. Further, unless otherwise specified, the terms used herein are interpreted in the meaning commonly used in the art.

### <Method for Synthesizing Double-Stranded DNA>

The double-stranded DNA synthesis method of the present invention is characterized in that it is a method for obtaining a double-stranded DNA fragment of interest by connecting short double-stranded DNAs to each other by overlap extension PCR, wherein the method has a plurality of stages of annealing temperature setting in a PCR cycle of the overlap extension PCR. The double-stranded DNA synthesis method of the present invention, by having a plurality of stages of annealing temperature setting in the PCR cycle, becomes applicable to the bonding of a plurality of DNAs having different Tm values, thus the method of the present invention can assemble a large number of single-stranded DNAs having homologous regions easily, accurately, efficiently, and quickly. Further, since the double-stranded DNA synthesis method of the present invention is applicable to the bonding of a plurality of DNAs having different Tm values, the method does not require labor to optimize the sequences of single-stranded oligo DNAs used as the materials or does not need to delicately reset the temperature for each double-stranded DNA fragment of interest. Furthermore, the method of the present invention can synthesize a double-stranded DNA fragment of interest easily, accurately, efficiently, and quickly, regardless of the sequence thereof, so that even a double-stranded DNA fragment having a conventional complex sequence (for example, a long repeat sequence, a sequence of contiguous identical nucleotides, or AT-rich or GC-rich sequence), which has been hitherto difficult or impossible to synthesize, can be synthesized.

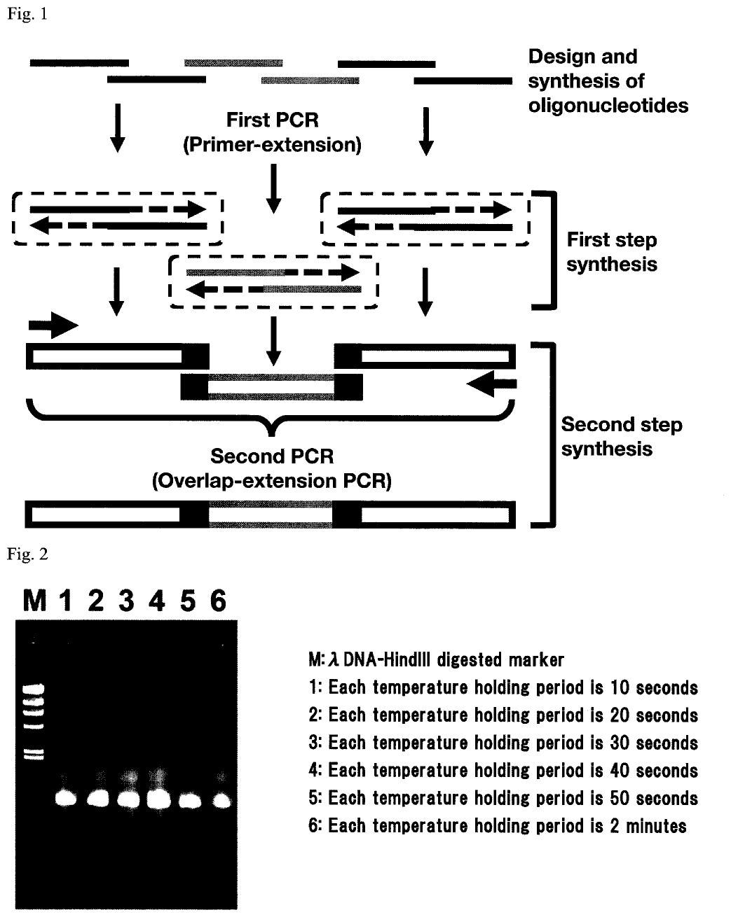

It is preferable that the short double-stranded DNAs used in the overlap extension PCR are synthesized by primer extension PCR. Accordingly, the method of the present invention can be described as a method for synthesizing a double-stranded DNA characterized by comprising a primer extension PCR step of synthesizing short double-stranded DNAs by primer extension PCR, and an overlap extension PCR step of connecting the double-stranded DNAs synthesized in the primer extension PCR step to each other by overlap extension PCR to synthesize a double-stranded DNA fragment of interest, wherein the method having a plurality of stages of annealing temperature setting in a PCR cycle of the overlap extension PCR. Hereinafter, the double-stranded DNA synthesis method of the present invention will be described for each step. FIG. 1 schematically shows the steps of the double-stranded DNA synthesis method of the present invention.

### [Primer Extension PCR Step]

This step is a step of synthesizing short double-stranded DNAs by primer extension PCR. The short double-stranded DNAs obtained in this step are connected to each other in the overlap extension PCR step described later to become a double-stranded DNA fragment, which can be used as an assembly material for synthesizing a long chain DNA. Thus, from the sequence of the long chain double-stranded DNA to be finally synthesized, it is determined what double-stranded DNA fragments are necessary as the assembly materials, and then the determined double-stranded DNA fragments are divided and designed into some short double-stranded DNAs which are to be synthesized in this step.

As used herein, the primer extension PCR in the present invention refers to a reaction to synthesize a double-stranded DNA by bonding a pair of single-stranded oligo DNAs having a region complementarily binding to a terminal portion of each other, and extending each chain using a DNA polymerase.

### (i) Sequence Design of Single-Stranded Oligo DNA

The full-length sequence of the double-stranded DNA fragment to be obtained by the double-stranded DNA synthesis method of the present invention is divided into an optional number of short double-stranded DNAs. The size of each of the short double-stranded DNAs is usually 100 to 300 bases, preferably 120 to 250 bases, more preferably 140 to 180 bases, further preferably around 150 bases. The dividing number into short double-stranded DNAs can be appropriately determined depending on the length of the full-length sequence, but it is usually 3 to 20, preferably 2 to 10, more preferably 2 to 5, and further preferably about 3.

The short double-stranded DNA which is obtained by the dividing is designed to include a mutually overlapping region in any length at the 5′-or 3′-end. The length of the overlapping region is usually 5 to 50 bases, preferably 10 to 40 bases, more preferably 15 to 40 bases, and further preferably around 30 bases. A pair of single-stranded oligo DNAs used in the synthesis of the short double-stranded DNA which is designed as described above is 60 to 300 bases including the overlapping region, preferably 100 to 200 bases, and more preferably 150 bases to 200 bases. In this step, by using a single-stranded oligo DNA having a relatively long chain, it is possible to reduce the number of single-stranded oligo DNA used in the synthesis of the double-stranded DNA while it is capable of eliminating the deviation of GC content ratio in the single-stranded oligo DNA sequence, thus it is also possible to prevent the formation of DNA secondary structure such as hairpin structure. It should be noted that these single-stranded oligo DNAs can be prepared by chemical synthesis.

These single-stranded oligo DNAs may include a single-stranded oligo DNA of incomplete-length generated during the process of chemical synthesis, which is different from the single-stranded oligo DNA of complete-length. In this step, when the content of single-stranded oligo DNA of complete-length is 15% or more in the single-stranded oligo DNA to be used, the DNA amplification product of the synthesized double-stranded DNA of interest can be obtained, and by cloning, it is possible to obtain a double-stranded DNA of accurate sequence. That is, even in a case where the single-stranded oligo DNA to be used contains about 85% of single-stranded oligo DNAs of incomplete-length including unreacted substances, it is possible to obtain a DNA amplification product of the synthesized double-stranded DNA of interest.

### (ii) PCR Cycle

A set number of pairs of single-stranded oligo DNAs, where the set number is corresponding to the dividing number described above, are prepared separately at any concentration in sterile water or TE buffer or the like. The concentration is usually 0.1 to 10 μM, preferably 0.25 to 5 μM, and more preferably around 1 μM. PCR reaction solutions (1× Phusion HF Buffer, 0.2 mM dNTP, 0.016 U/μL Phusion High-Fidelity DNA polymerase; New England Biolabs Japan Inc. and the like) are prepared, and each pair of single-stranded oligo DNAs is added to one of the solutions so that total amount of the resultant solution become equal each other. In other words, the PCR reaction solutions are prepared for each short double-stranded DNA to be synthesized, and only one pair of two single-stranded oligo DNAs is added to one of the PCR reaction solutions, and mixed. Thus, the mismatching between single-stranded oligo DNAs and formation of the primer dimer which occur during the bonding of single-stranded oligo DNAs can be prevented.

Next, using a thermal cycler, primer extension PCR reaction is performed to synthesize a short double-stranded DNA. The temperature condition for the PCR reaction in this case is 98° C. for 2 minutes and subsequent an optional number of cycles with heat denaturation phase/annealing phase/elongation phase (extension) as one cycle. The number of cycles is usually 5 cycles or more, preferably 10 cycles or more. There is no problem even when the number of cycles is more than 10, but the synthesized amount obtained in about 10 cycles can be said enough in this step.

In the heat denaturation and elongation phases, the same conditions as that in conventional PCR methods can be selected. For example, in the heat denaturation phase, the condition may be 98° C. for 30 seconds. In the elongation phase, the condition varies depending on the DNA polymerase used in the phase, but, for example, may be 72° C. for 50 seconds.

In the method of the present invention, the annealing phase has a plurality of stages of annealing temperature setting, which is a significant difference from the conventional methods. The annealing temperature of the annealing temperature setting is in the range of 50 to 90° C., preferably in the range of 60 to 85° C., and the number of stages wherein the annealing temperature setting is performed is 2 to 20 stages, preferably 2 to 15 stages, more preferably 2 to 10 stages, and further preferably 2 to 8 stages. The holding period of the annealing temperature in each stage is usually 10 seconds or more, preferably 20 seconds or more, more preferably 30 seconds or more, further preferably 40 seconds or more, and particularly preferably 50 seconds or more. There is no problem even when the holding period exceeds 50 seconds and the reaction may continue up to about 2 minutes, but it can be said that about 50 seconds are sufficient. Note that there is no problem even when an overnight incubation is performed after the primer extension PCR step is completed. The temperature of the incubation is preferably in the range of 4 to 72° C.

By having a plurality of stages of annealing temperature setting in the PCR of this step, it is possible to complement the difference of Tm values in the combination of each pair of the single-stranded oligo DNAs, thus all PCR reactions for the double-stranded DNA synthesis in this step can be performed at one time.

### [Overlap Extension PCR Step]

This step is a step of connecting the short double-stranded DNAs synthesized in the primer extension PCR step to each other by overlap extension PCR to obtain a double-stranded DNA fragment of interest. It is a characteristic of this invention to have a plurality of stages of annealing temperature setting in this PCR cycle.

The overlap extension PCR as used herein refers to a reaction in which, upon amplification of the target DNA in PCR reaction, by adding another sequence to the 5′-end side of the target DNA specific primer, a new sequence is added to the PCR product, and by designing the new sequence in advance to be complementary among a plurality of the target DNAs, the ends of a plurality of the target DNAs are fused each other during annealing, and by performing a subsequent elongation reaction using a DNA polymerase, a PCR product is synthesized. In the present invention, the overlap extension PCR reaction is a reaction performed to generate a double-stranded DNA fragment by connecting a plurality of kinds of short double-stranded DNAs synthesized in the primer extension PCR step to each other. The short double-stranded DNA has a homology region at the connecting portion, and after it becomes a single-stranded by heat denaturation, the homologous region at the portions complementarily couple each other in the annealing phase, and new sequences at 5′ end side in both strands can be adhered to the PCR product. Since the new sequence is designed to be complementary to another double-stranded DNA, the ends of the DNAs can fuse each other during annealing, and by performing the subsequent elongation reaction using a DNA polymerase, a PCR product can be synthesized.

Specifically, the step is performed as follows. The short double-stranded DNAs synthesized in the primer extension PCR step each are added in equal amounts to one of PCR reaction solutions (1×Phusion HF Buffer, 0.2 mM dNTP, 0.016 U/μL Phusion High-Fidelity DNA polymerase; manufactured by New England Biolabs Japan Inc., and the like) which are solutions for synthesizing full-length double-stranded DNA. These PCR reaction solutions include a primer for amplifying the full-length double-stranded DNA.

Next, using a thermal cycler, PCR reaction is performed to synthesize the full-length double-stranded DNA fragment. The temperature condition for the PCR reaction in this case is 98° C. for 2 minutes and subsequent an optional number of cycles with the heat denaturation phase/annealing phase/elongation phase (extension) as one cycle. The number of cycles is usually 10 cycles or more, preferably 20 cycles or more. There is no problem even when the number of cycles is more than 20, but the synthesized amount obtained in about 20 cycles can be said enough in this step.

In the heat denaturation and elongation phases, the same conditions as that in conventional PCR methods can be selected. For example, in the heat denaturation phase, the condition may be 98° C. for 30 seconds. In the elongation phase, the condition varies depend on a DNA polymerase used, but, for example, may be 72° C. for 50 seconds.

In the method of the present invention, the annealing phase has a plurality of stages of annealing temperature setting, which is a significant difference from the conventional methods. The annealing temperature of the annealing temperature setting is in the range of 50 to 90° C., preferably in the range of 60 to 85° C., and the number of stages wherein the annealing temperature setting is performed is 2 to 20 stages, preferably 2 to 15 stages, more preferably 2 to 10 stages, and further preferably 2 to 8 stages. The holding period of the annealing temperature in each stage is usually 10 seconds or more, preferably 20 seconds or more, more preferably 30 seconds or more, further preferably 40 seconds or more, and particularly preferably 50 seconds or more. There is no problem even when the holding period exceeds 50 seconds and the reaction may continue up to about 2 minutes, but it can be said that about 50 seconds are sufficient.

By having a plurality of stages of annealing temperature setting in PCR of this step, it is possible to complement the difference of Tm values which occurs during assembly of short double-stranded DNAs or when bonding the single-stranded DNAs each other, thus it is not required to optimize the Tm value to match the homologous region of the single-stranded oligo DNA necessary for bonding. Therefore, the length of the homologous region of the single-stranded oligo DNA can be designed in a state fixed to, for example, 30 bases, and, when synthesizing a large number of double-stranded DNAs having different sequences, one can easily design a single-stranded oligo DNA. It is also possible to unify the respective temperature conditions of the primer extension PCR reaction and the overlap extension PCR reaction.

Furthermore, according to the synthesis method of the present invention, it is possible to simultaneously synthesize a plurality of double-stranded DNA fragments having different sequences in parallel. The number of double-stranded DNA fragments which can be simultaneously synthesized is not particularly limited, but it is possible to simultaneously synthesize double-stranded DNA fragments of several ten to several hundred units, which indicates that the throughput property is significantly high. Further, it is also possible to automate the simultaneous synthesis of a plurality of double-stranded DNA fragments by applying a synthesis method of the present invention to a program used in a liquid dispensing robot.

The sequence and length of the double-stranded DNA fragment which can be synthesized by the synthesis method of the present invention are not particularly limited, but in light of the accuracy of the thermostable DNA polymerase used in PCR, it is preferable that the length is up to 5,000 base pairs. Therefore, the number of short double-stranded DNA fragments to be connected to each other by overlap extension PCR reaction is usually about 3 to 20 fragments, preferably 2 to 10 fragments, more preferably 2 to 5 fragments, and further preferably around 3 fragments.

Further, according to the synthesis method of the present invention, it becomes possible to synthesize a double-stranded DNA fragment comprising a DNA sequence generally considered as a complex sequence such as a long repeat sequence, a sequence of contiguous identical nucleotides, or an AT-rich or GC-rich sequence.

The double-stranded DNA fragment obtained in the synthesis method of the present invention can be assembled in large numbers and constructed to a long-chain DNA by using long-chain DNA synthesis method such as a yeast assembly method (see Gibson, D. G., et al. Science, 5867, 1215-1220., 2008), Gibson assembly method (see Gibson, D. G., et al. Nat. Methods, 6, 343-345., 2009), Golden gate method (see Engler, C., et al. PLoS ONE, 4, e5553., 2009), LCR method (see Stefan de Kok, S., et al. ACS Synth. Biol., 3, 97-106., 2014), and OGAB method (see Tsuge, K., et al. Nucleic Acids Res., 31, e133., 2003, Tsuge, K., et al. Sci. Rep., 5, 10655., 2015).

In the synthesis of long-chain DNA, double-stranded DNA fragments are used as the assembly materials, but even the lack of one double-stranded DNA fragment makes it impossible to synthesize the long chain DNA of interest. The synthesis method of the present invention, regardless of the complexity of the DNA sequence of interest to be synthesized, can simultaneously synthesize a plurality of double-stranded DNA fragments, so that it can quickly synthesize double-stranded DNA fragments which become assembly materials for synthesizing a long chain DNA. Therefore, the synthesis method of the present invention can supply quickly double-stranded DNA fragments necessary for synthesizing a long-chain DNA, and thus can significantly improve the throughput property for the synthesis method of long chain DNA.

The PCR product of the double-stranded DNA fragments obtained by the synthetic method of the present invention is mixed with a vector, and subjected to DNA cloning by DNA ligation reaction, and then applied to transformation using an E. coli competent cell with introduction of a plasmid. The method of DNA cloning of the DNA amplification product of the synthesized double-stranded DNA fragments is not particularly limited, and examples thereof include a restriction enzyme cloning method, a TA cloning method, an In-Fusion cloning method, and a blunt end cloning method. The vector DNA used is not particularly limited, and examples thereof include a restriction enzyme cloning vector, an In-Fusion cloning vector, a TA cloning vector and a blunt end cloning vector.

The DNA amplification product of double-stranded DNA fragments obtained by the synthesis method of the present invention can suppress the occurrence of non-specific DNA amplification products, which are shorter in the length than DNA sequence of interest, generated by PCR reaction. Thus, the synthesized double-stranded DNA fragments obtained in the synthesis method of the present invention can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained.

### <Program>

The present invention also includes a program for PCR in the double-stranded DNA synthesis method of the present invention described above. The program for PCR conditions of the present invention can be used in a device frequently used for PCR such as a thermal cycler. The program of the present invention defines the temperature conditions or the like of PCR in the double-stranded DNA synthesis method of the present invention, and the details of the content thereof can refer to the explanation in the section “Method for synthesizing double-stranded DNA”.

### <Device>

The present invention also includes a device which can realize the double-stranded DNA synthesis method of the present invention described above. Further, the device of the present invention is a device into which the program of the present invention is incorporated. Specifically, the device is a device frequently used for PCR such as a thermal cycler into which the program of the present invention is incorporated. The program of the present invention defines the temperature conditions of PCR or the like of the DNA synthesis method of the present invention, and the details of the content thereof can refer to the explanation in the section “Method for synthesizing double-stranded DNA”.

### <Double-Stranded DNA Automatic Synthesis System>

The present invention also includes a double-stranded DNA automatic synthesis system characterized by using the double-stranded DNA synthesis method of the present invention described above. In the double-stranded DNA synthesis method of the present invention, PCR can be performed in a consistent condition to synthesize a double-stranded DNA, regardless of the sequence of the DNA of interest, thus it is possible that the system of the present invention is used as a high throughput synthesis system using liquid dispensing robot or the like and an automated synthesis system which can perform the series of steps automatically. The double-stranded DNA automatic synthesis system of the present invention may be configured to use the program of the present invention described above and/or the device of the present invention.

### EXAMPLES

The present invention will be specifically described in the following Examples, but the present invention is not limited to the Examples.

The reagents, the test methods or the like commonly used in the Examples are as follows.

The single-stranded oligo DNAs used as materials for synthesizing a double-stranded DNA were manufactured by NIHON TECHNO SERVICE CO., LTD. and FASMAC. DNA amplification by PCR reaction for the double-stranded DNA synthesis was performed using Phusion High-Fidelity DNA polymerase from New England Biolabs Japan Inc. in accordance with the attached instruction. DNA cloning of DNA amplification products was performed using 10× A-attachment Mix manufactured by TOYOBO CO., LTD., and T-Vector pMD19 (Simple) and DNA Ligation kit manufactured by Takara Bio Inc. The Escherichia coli competent used was E. coli JM109 competent cell manufactured by Takara Bio Inc., and the transformation method using the plasmid vector was performed in accordance with the attached instruction. Media components in LB medium and agar used were manufactured by Becton, Dickinson and Company (Bacto™ Tryptone, Bacto™ Yeast Extract, Bacto™ Agar). Antibiotics ampicillin and carbenicillin were purchased from Nacalai Tesque, Inc. Preparation of templates for PCR reaction from E. coli colonies was performed using Cica geneus DNA extraction reagents manufactured by Kanto Chemical Co., Inc. in accordance with the attached instruction. DNA amplification by colony direct PCR reactions from E. coli colonies was performed using TaKaRa Ex-Taq Hot Start manufactured by Takara Bio Inc. in accordance with the attached instruction. Temperature conditions for colony direct PCR reactions were as follows: 95° C. for 2 minutes and subsequent 30 cycles of the following temperature cycles; 95° C. for 20 seconds; 58° C. for 30 seconds; 72° C. for 1 minute per amplification length of 1 kb. DNA purification was performed using MinElute PCR Purification kit manufactured by QIAGEN K.K. in accordance with the attached instruction. DNA sequencing reaction was performed using BigDye Terminator v3.1 Cycle Sequencing Kit manufactured by Thermo Fisher Scientific Inc. in accordance with the attached instruction. Temperature conditions for DNA sequencing reaction were as follows: 95° C. for 2 minutes and subsequent 30 cycles of the following temperature cycle: 95° C. for 5 seconds; 50° C. for 10 seconds; and 60° C. for 2 minutes and 30 seconds. Purification of DNA sequencing reaction products was performed using BigDye Terminator Purification kit manufactured by Thermo Fisher Scientific Inc. in accordance with the attached instruction. DNA sequencing was performed using Applied Biosystems 3500×L Genetic analyzer manufactured by Thermo Fisher Scientific Inc. in accordance with the attached instruction. Agarose gel electrophoresis of PCR reaction products was performed using an agarose gel electrophoresis device (i-MyRun. NC) manufactured by Cosmo Bio Co., Ltd. in accordance with the attached instruction. DNA staining after electrophoresis was performed using a GelRed nucleic acid fluorescence staining reagent manufactured by Biotium, Inc. in accordance with the attached instruction. Electrophoresis of single-stranded oligo DNA was performed using XCell SureLock Mini-Cell electrophoresis device and 10% Novex TBE-Urea gel, each manufactured by Thermo Fisher Scientific Inc. in accordance with the attached instruction. Staining of single-stranded oligo DNA after electrophoresis was performed using a SYBR Green II nucleic acid fluorescence staining reagent manufactured by Takara Bio Inc. in accordance with the attached instruction. Analysis of single-stranded oligo DNA was performed using a gel imaging analyzer Gel Doc EZ system manufactured by Bio-Rad Laboratories, Inc. in accordance with the attached instruction. All other biochemical reagents used were products of Thermo Fisher Scientific Inc. and Nacalai Tesque, Inc.

### (Example 1) Study of PCR Reaction Condition in the Synthesis Method of the Present Invention (Study of Temperature Holding Period of Each temperature Gradient in a Plurality of Stages)

### (1) Sequence Design of Single-Stranded Oligo DNA

The sequence of full-length double-stranded DNA of interest was divided into three short double-stranded DNA fragments. These short double-stranded DNA fragments were designed so that the overlapping region of 30 base pairs was contained in the 5′- or 3′-end. In other words, six single-stranded oligo DNAs for synthesizing three short double-stranded DNA fragments were designed to have a length of about 150 bases containing an overlapping region of 30 bases (SEQ ID NOS: 1 to 6). The forward primer (SEQ ID NO: 7) and the reverse primer (SEQ ID NO: 8) for the overlap extension PCR reaction were also designed.

### (2) Two-Step Type Double-Stranded DNA Synthesis

The six single-stranded oligo DNAs required for synthesizing a double-stranded DNA were prepared at a concentration of 1 μM in sterile water or TE buffer (manufactured by Nacalai Tesque, Inc.). For synthesizing short double-stranded DNA fragments by the primer extension PCR, three PCR reaction solutions A (1×Phusion HF Buffer, 0.2 mM dNTP, 0.016 U/μL Phusion High-Fidelity DNA polymerase) were prepared, and then, to the first PCR reaction solution A, each 1 μL of single-stranded oligo DNA (SEQ ID NO: 1) and single-stranded oligo DNA (SEQ ID NO: 2) was added; to the second PCR reaction solution A, each 1 μL of single-stranded oligo DNA (SEQ ID NO: 3) and single-stranded oligo DNA (SEQ ID NO: 4) was added; and to the third PCR reaction solution A, each 1 μL of single-stranded oligo DNA (SEQ ID NO: 5) and single-stranded oligo DNA (SEQ ID NO: 6) was added. Each solution was prepared so that the total amount become 25 μL. PCR reaction solutions B (1×Phusion HF Buffer, 0.2 mM dNTP, 0.016 U/μL. Phusion High-Fidelity DNA polymerase, 0.1 μM forward primer: SEQ ID NO: 7, 0.1 μM reverse primer: SEQ ID NO: 8) for synthesizing a full-length double-stranded DNA by overlap extension PCR were prepared. Each solution was prepared so that the total amount become 25 μL.

Using a thermal cycler manufactured by Takara Bio Inc., three short double-stranded DNA fragments were synthesized by primer extension PCR reaction. The temperature condition for PCR reaction in this case was as follows.

98° C. for 2 minutes and subsequent 10 cycles of the following temperature cycle:

### (Heat Denaturation Phase)

98° C. for 30 seconds;

### (Annealing Phase)

77.5° C. for 10 seconds; 75° C. for 10 seconds; 72.5° C. for 10 seconds; 70° C. for 10 seconds; 67.5° C. for 10 seconds; 65° C. for 10 seconds; 62.5° C. for 10 seconds;

### (Elongation Phase)

72° C. for 50 seconds.

Thereafter, to the PCR reaction solution B maintaining at 72° C., each 1 μL of the three PCR reaction solutions A of the short double-stranded DNA was added.

Next, using a thermal cycler manufactured by Takara Bio Inc., the three short double-stranded DNA fragments prepared above were connected to each other in multistage by overlap extension PCR reaction to synthesize a full-length double-stranded DNA. The temperature condition for PCR reaction in this case was as follows.

98° C. for 2 minutes and subsequent 20 cycles of the following temperature cycle:

### (Heat Denaturation Phase)

98° C. for 30 seconds;

### (Annealing Phase)

77.5° C. for 10 seconds; 75° C. for 10 seconds; 72.5° C. for 10 seconds; 70° C. for 10 seconds; 67.5° C. for 10 seconds; 65° C. for 10 seconds; 62.5° C. for 10 seconds;

### (Elongation Phase)

72° C. for 50 seconds.

Thereafter, the resultant was treated at 72° C. for 3 minutes.

Except that all of the temperature holding period in the stages of the annealing phase were 20 seconds, 30 seconds, 40 seconds, 50 seconds or 2 minutes, a double-stranded DNA of the full-length was synthesized respectively under the same PCR conditions as described above. Thereafter, each portion of the DNA amplification products of the double-stranded DNA fragments obtained from 6 conditions in the synthesis method of the present invention (the temperature holding period in the stages of the annealing phase is 10 seconds, 20 seconds, 30 seconds, 40 seconds, 50 seconds, or 2 minutes) was electrophoresed on a 1% agarose gel using an agarose gel electrophoresis device (Cosmo Bio Co., Ltd.). The results are shown in FIG. 2.

As a result, the DNA amplification product of the double-stranded DNA fragments obtained when the temperature holding period of the PCR reaction condition was 50 seconds or more in the synthesis method of the present invention was able to suppress the occurrence of non-specific DNA amplification products (DNA amplification products shorter in the length than DNA sequence of interest) lower than the DNA amplification product of the double-stranded DNA fragments obtained when the temperature holding period of the PCR reaction condition was less than 50 seconds. That is, it is particularly preferable that the temperature holding period of the PCR reaction condition in the synthesis method of the present invention is 50 seconds or more. It is possible to obtain the double-stranded DNA fragment of interest from the DNA amplification product of the double-stranded DNA fragment obtained in the PCR reaction condition that the temperature holding period is 10 seconds, 20 seconds, 30 seconds, or 40 seconds, however it contains non-specific DNA amplification products. When the temperature holding period of the PCR reaction condition of the synthesis method of the present invention is 50 seconds or more, it is possible to suppress the occurrence of non-specific DNA amplification product as described above, thus the DNA amplification product of double-stranded DNA fragments can be applied to DNA cloning methods such as a TA cloning method and a blunt end cloning method, and thus the cloned DNA containing the double-stranded DNA fragment of interest can be quickly obtained.

### (Example 2) Study of PCR Reaction Conditions of the Synthesis Method of the Present Invention (Study of the Number of Stages in the Annealing)

### (1) Sequence Design of Single-Stranded Oligo DNA

The sequence of full-length double-stranded DNA of interest was divided into three short double-stranded DNA fragments. These short double-stranded DNA fragments were designed so that the overlapping region of 30 base pairs was contained in the 5′- or 3′-end. In other words, six single-stranded oligo DNA for synthesizing three short double-stranded DNA fragments were designed to have a length of about 150 bases containing an overlapping region of 30 bases (SEQ ID NOS: 9 to 14). The forward primer (SEQ ID NO: 15) and the reverse primer (SEQ ID NO: 16) for the overlap extension PCR reaction were also designed.

### (2) Two-Step Type Double-Stranded DNA Synthesis

The six single-stranded oligo DNAs required for synthesizing a double-stranded DNA were prepared at a concentration of 1 μM in sterile water or TE buffer (manufactured by Nacalai Tesque, Inc.). For synthesizing short double-stranded DNA fragments, three PCR reaction solutions A (1×Phusion HF Buffer, 0.2 mM dNTP, 0.016 U/μL Phusion High-Fidelity DNA polymerase) were prepared, and then, to the first PCR reaction solution A, each 1 μL of single-stranded oligo DNA (SEQ ID NO: 9) and single-stranded oligo DNA (SEQ ID NO: 10) was added; to the second PCR reaction solution A, each 1 μL of single-stranded oligo DNA (SEQ ID NO: 11) and single-stranded oligo DNA (SEQ ID NO: 12) was added; and to the third PCR reaction solution A, each 1 μL of single-stranded oligo DNA (SEQ ID NO: 13) and single-stranded oligo DNA (SEQ ID NO: 14) was added. Each solution was prepared so that the total amount become 25 μL. PCR reaction solutions B (1×Phusion HF Buffer, 0.2 mM dNTP, 0.016 U/μL Phusion High-Fidelity DNA polymerase, 0.1 μM forward primer: SEQ ID NO: 15, 0.1 μM reverse primer: SEQ ID NO: 16) for synthesizing a full-length double-stranded DNA was prepared. Each solution was prepared so that the total amount become 25 μL.

The double-stranded DNA fragments of full-length were synthesized in the conditions (i) to (vii) below (the number of stages of the annealing temperature settings are different).

### (i) 0 Stages

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 72° C. for 50 seconds.

Thereafter, to the PCR reaction solution B maintaining at 72° C., each 1 μL of the three PCR reaction solutions A of the short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 72° C. for 50 seconds.

Thereafter, the resultant was treated at 72° C. for 3 minutes.

### (ii) 1 Stage

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 72° C. for 50 seconds.

Thereafter, to the PCR reaction solution B maintaining at 72° C., each 1 μL of the three PCR reaction solutions A of the short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 77.5° C. for 50 seconds; 72° C. for 50 seconds.

Thereafter, the resultant was treated at 72° C. for 3 minutes.

(iii) 2 Stages

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 62.5° C. for 50 seconds; 72° C. for 50 seconds.

Thereafter, to the PCR reaction solution B maintaining at 72° C., each 1 μL of three PCR reaction solutions A of short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 77.5° C. for 50 seconds; 62.5° C. for 50 seconds; 72° C. for 50 seconds.

Thereafter, the resultant was treated at 72° C. for 3 minutes.

### (iv) 3 Stages

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 70° C. for 50 seconds; 62.5° C. for 50 seconds; 72° C. for 50 seconds.

Thereafter, to the PCR reaction solution B maintaining at 72° C., each 1 μL of three PCR reaction solutions A of short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle: 98° C. for 30 seconds; 77.5° C. for 50 seconds; 70° C. for 50 seconds; 62.5° C. for 50 seconds; 72° C. for 50 seconds.

Thereafter, the resultant was treated at 72° C. for 3 minutes.

### (v) 7 Stages

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., each 1 μL of three PCR reaction solutions A of short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

### (vi) 8 Stages

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75.4° C. for 50 seconds; 73.2° C. for 50 seconds; 71.1° C. for 50 seconds; 68.9° C. for 50 seconds; 66.8° C. for 50 seconds; 64.6° C. for 50 seconds; 62.5° C. for 50 seconds; 72° C. for 50 seconds.

Thereafter, to the PCR reaction solution B maintaining at 72° C., each 1 μL of three PCR reaction solutions A of short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 77.5° C. for 50 seconds; 75.4° C. for 50 seconds; 73.2° C. for 50 seconds; 71.1° C. for 50 seconds; 68.9° C. for 50 seconds; 66.8° C. for 50 seconds; 64.6° C. for 50 seconds; 62.5° C. for 50 seconds; 72° C. for 50 seconds.

Thereafter, the resultant was treated at 72° C. for 3 minutes.

(vii) Continuous Temperature Gradient

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. to 62.5° C. at 0.04° C./seconds; 72° C. for 50 seconds.

Thereafter, to the PCR reaction solution B maintaining at 72° C., each 1 μL of three PCR reaction solutions A of short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 77.5° C. to 62.5° C. at 0.04° C./seconds; 72° C. for 50 seconds

Thereafter, the resultant was treated at 72° C. for 3 minutes.

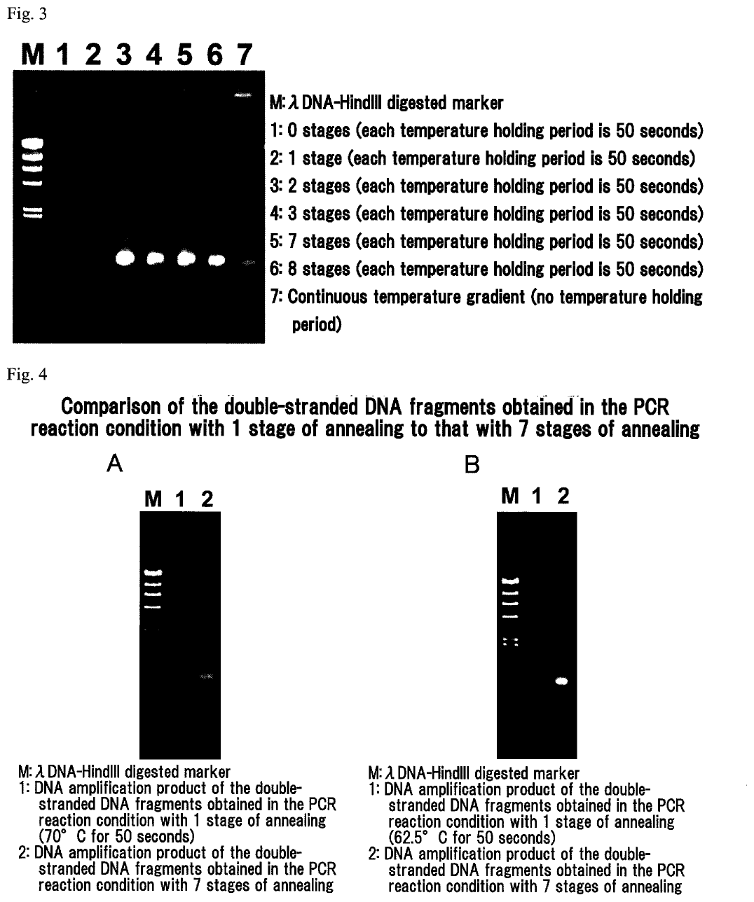

Thereafter, a portion of DNA amplification product of the double-stranded DNA fragments obtained from 7 conditions in the synthesis method of the present invention were electrophoresed on a 1% agarose gel using an agarose gel electrophoresis device (Cosmo Bio Co., Ltd.). The results are shown in FIG. 3.

As shown in FIG. 3, it is preferred that the annealing temperature setting of the PCR reaction condition in the synthesis method of the present invention is performed at 2 to 8 stages. There is no problem that the number of stages of the annealing temperature setting is 8 stages or more, and the reaction may be performed under the PCR reaction condition of the temperature gradient of 8 stages or more, but it can be said that the number of stages is sufficient at about 2 to 8 stages. On the other hand, it has been confirmed that, in the annealing temperature setting of the PCR reaction conditions of the synthesis method of the present invention, when there is no temperature holding period (continuous temperature gradient) or the temperature holding period is very short, it is difficult to obtain the DNA amplification product of the double-stranded DNA fragment of interest, even when the number of stages of the annealing temperature setting is increased (data not shown).

### (Example 3) Study of PCR Reaction Conditions of the Synthesis Method of the Present Invention (Comparison of the Double-Stranded DNA Fragment Obtained in the PCR Reaction Condition with an Annealing Temperature Setting of 1 Stage to that with an Annealing Temperature Setting of 7 Stages)

### (1) Sequence Design of Single-Stranded Oligo DNA

The sequence was designed in the same manner as in Example 2.

### (2) Two-Step Type Double-Stranded DNA Synthesis

The synthesis of the double-stranded DNA was performed in the same manner as in Example 2.

(i) Comparison of the DNA Amplification Product of Double-Stranded DNA Fragments Obtained in the PCR Reaction Condition with 1 Stage of Annealing temperature Setting to that with 7 Stages of Annealing Temperature Setting-1
(a) DNA Amplification Product of Double-Stranded DNA Fragments Obtained in the PCR Reaction Condition with 1 Stage of Annealing Temperature Setting

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 70° C. for 50 seconds; 72° C. for 50 seconds.

Thereafter, to the PCR reaction solution B maintaining at 72° C., each 1 μL of the three PCR reaction solutions A of the short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 70° C. for 50 seconds; 72° C. for 50 seconds.

Thereafter, the resultant was treated at 72° C. for 3 minutes.

(b) DNA Amplification Product of Double-Stranded DNA Fragments Obtained in the PCR Reaction Condition with 7 Stages of Annealing Temperature Setting

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., each 1 μL of the three PCR reaction solutions A of the short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

(ii) Comparison of the DNA Amplification Product of Double-Stranded DNA Fragments Obtained in the PCR Reaction Condition with 1 Stage of Annealing Temperature Setting to that with 7 Stages of Annealing Temperature Setting-2
(a) DNA Amplification Product of Double-Stranded DNA Fragments Obtained in the PCR Reaction Condition with 1 Stage of Annealing Temperature Setting

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 62.5° C. for 50 seconds; 72° C. for 50 seconds.

Thereafter, to the PCR reaction solution B maintaining at 72° C., each 1 μL of the three PCR reaction solutions A of the short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The PCR reaction condition in this case was 98° C. for 2 minutes and subsequent 20 cycles of the following temperature cycle:

98° C. for 30 seconds; 62.5° C. for 50 seconds; 72° C. for 50 seconds.

Thereafter, the resultant was treated at 72° C. for 3 minutes.

(b) DNA Amplification Product of Double-Stranded DNA Fragments Obtained in the PCR Reaction Condition with 7 Stages of Annealing Temperature Setting

The DNA amplification product of double-stranded DNA fragments was obtained in the same method as in the section “(b) DNA amplification product of double-stranded DNA fragments obtained in the PCR reaction condition with 7 stages of annealing temperature setting” in “(i) Comparison of the DNA amplification product of double-stranded DNA fragments obtained in the PCR reaction condition with 1 stage of annealing temperature setting to that with 7 stages of annealing temperature setting-1”.

Thereafter, portions of the DNA amplification products of double-stranded DNA fragments obtained from each PCR condition described above in the synthesis method of the present invention were electrophoresed on a 1% agarose gel using an agarose gel electrophoresis device (Cosmo Bio Co., Ltd.). The results are shown in FIGS. 4A and B.

As shown in FIGS. 4A and B, in the PCR reaction condition where the annealing temperature setting was performed at 7 stages, the DNA amplification product of the double-stranded DNA fragment was obtained clearly. On the other hand, in the PCR reaction condition where the annealing temperature setting was performed at 1 stage (the annealing temperature was 70° C. or 62.5° C.), the DNA amplification product of the double-stranded DNA fragment could not be obtained. From the above results, as shown in Example 2 described above, it is difficult to synthesize a double-stranded DNA sequence when the annealing temperature setting is performed at 1 stage or less in the PCR reaction condition in the synthesis method of the present invention, while it is possible to synthesize double-stranded DNA sequence when the annealing temperature setting is performed at 2 stages or more. Thus, in the PCR reaction condition in the synthesis method of the present invention, it is preferred that annealing temperature setting is performed at 2 to 8 stages.

### (Example 4) Comparison of the Double-Stranded DNA Fragments Obtained by the Conventional Synthesis Method to that Obtained by the SYNTHESIS METHOD of the Present Invention-1

(Comparison of the Double-Stranded DNA Fragments Obtained by the Synthesis Method of the Synthesis Trustee Company to that Obtained by the Synthesis Method of the Present Invention)

### (1) Sequence Design of Single-Stranded Oligo DNA

The sequence of full-length double-stranded DNA of interest was divided into three short double-stranded DNA fragments. These short double-stranded DNA fragments were designed so that the overlapping region of 30 base pairs was contained in the 5′- or 3′-end. In other words, six single-stranded oligo DNAs for synthesizing three short double-stranded DNA fragments were designed to have a length of about 150 bases containing an overlapping region of 30 bases (SEQ ID NOS: 17 to 22). The forward primer (SEQ ID NO: 23) and the reverse primer (SEQ ID NO: 24) for the overlap extension PCR reaction were also designed.

### (2) Two-Step Type Double-Stranded DNA Synthesis

The six single-stranded oligo DNAs required for synthesizing a double-stranded DNA were prepared at a concentration of 1 μM in sterile water or TE buffer (manufactured by Nacalai Tesque, Inc.). For synthesizing short double-stranded DNA fragments, three PCR reaction solutions A (1×Phusion HF Buffer, 0.2 mM dNTP, 0.016 U/μL Phusion High-Fidelity DNA polymerase) were prepared, and then, to the first PCR reaction solution A, each 1 μL of single-stranded oligo DNA (SEQ ID NO: 17) and single-stranded oligo DNA (SEQ ID NO: 18) was added; to the second PCR reaction solution A, each 1 μL of single-stranded oligo DNA (SEQ ID NO: 19) and single-stranded oligo DNA (SEQ ID NO: 20) was added; and to the third PCR reaction solution A, each 1 μL of single-stranded oligo DNA (SEQ ID NO: 21) and single-stranded oligo DNA (SEQ ID NO: 22) was added. Each solution was prepared so that the total amount become 25 μL. PCR reaction solutions B (1×Phusion HF Buffer, 0.2 mM dNTP, 0.016 U/μL Phusion High-Fidelity DNA polymerase, 0.1 μM forward primer: SEQ ID NO: 23, 0.1 μM reverse primer : SEQ ID NO: 24) for synthesizing a full-length double-stranded DNA were prepared. Each solution was prepared so that the total amount become 25 μL.

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., each 1 μL of the three PCR reaction solutions A of the short double-stranded DNA was added.

Further, using a thermal cycler manufactured by Takara Bio Inc., the three short double-stranded DNA fragments were connected to each other in multistage by overlap extension PCR reaction to synthesize a full-length double-stranded DNA. The temperature condition for the PCR reaction in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds ;62.5° C. for 50 seconds; 72° C. for 50 seconds.

Thereafter, the resultant was treated at 72° C. for 3 minutes.

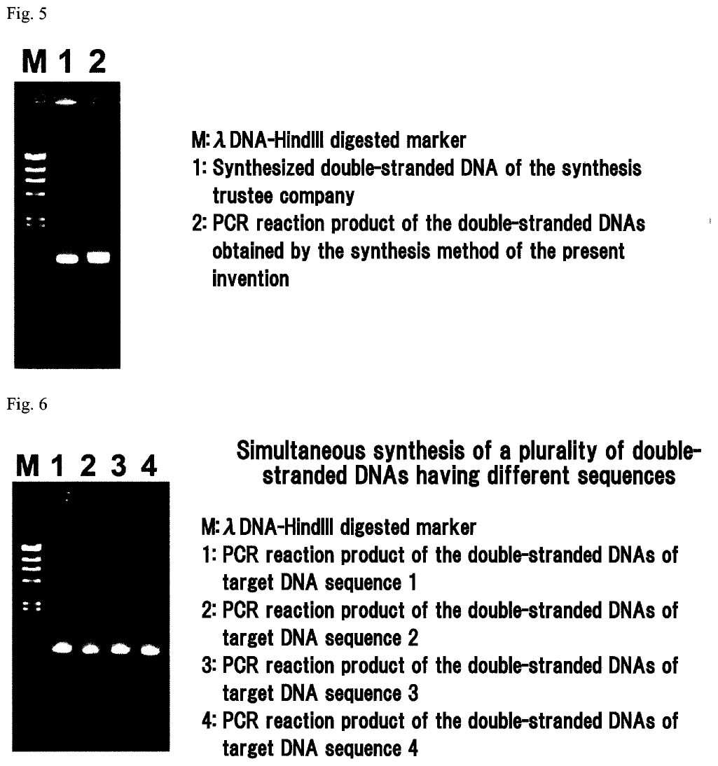

A portion of the DNA amplification product of the synthesized double-stranded DNA described above obtained by the method of the present invention and a portion of the solution of the double-stranded DNA synthesized by the requested synthesis trustee company (according to conventional synthetic methods) were electrophoresed on a 1% agarose gel using an agarose gel electrophoresis device (Cosmo Bio Co., Ltd.). The results are shown in FIG. 5.

As shown in FIG. 5, the DNA amplification product of double-stranded DNA fragments obtained by the synthesis method of the present invention was able to suppress the occurrence of non-specific DNA amplification products (DNA amplification products shorter in the length than DNA sequence of interest) more significantly than the DNA amplification product of double-stranded DNA fragments obtained by the synthesis method of the synthesis trustee company. The DNA amplification product of double-stranded DNA fragments of the synthesis trustee company in which a large amount of non-specific DNA amplification product occurred is not suitable for applying to DNA cloning methods such as a TA cloning method and a blunt end cloning method, because it is necessary to go through a step of cutting out purification of a gel or other steps to take out only the double-stranded DNA fragment of interest, thus it is difficult to obtain cloned DNA quickly.

On the other hand, the DNA amplification product of double-stranded DNA fragments obtained by the synthesis method of the present invention was able to suppress the occurrence of non-specific DNA amplification products (DNA amplification products shorter in the length than DNA sequence of interest) significantly. Thus, the DNA amplification product of double-stranded DNA fragments obtained in the PCR reaction condition in the synthesis method of the present invention can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained. From the result above, it can be said that the synthesis method of the present invention is superior to the conventional DNA synthesis methods in that it increases the efficiency of DNA cloning and is possible to quickly obtain cloned DNA of interest.

### (Example 5) Analysis of Simultaneous Synthesis of a Plurality of Double-Stranded DNAs having Different Sequences and Chemically Synthesized Single-Stranded Oligo DNA

### (1) Sequence Design of Single-Stranded Oligo DNA

The four sequences of full-length double-stranded DNA of interest were each divided into three short double-stranded DNA fragments respectively. These short double-stranded DNA fragments were designed so that the overlapping region of 30 base pairs was contained in the 5′- or 3′-end. Six single-stranded oligo DNAs for synthesizing three short double-stranded DNA fragments corresponding to the first full-length double-stranded DNA were designed to have a length of about 150 bases containing an overlapping region of 30 bases (SEQ ID NOS: 25 to 30). Six single-stranded oligo DNAs for synthesizing three short double-stranded DNA fragments corresponding to the second full-length double-stranded DNA were designed to have a length of about 150 bases containing an overlapping region of 30 bases (SEQ ID NOS: 31 to 36). Six single-stranded oligo DNAs for synthesizing three short double-stranded DNA fragments corresponding to the third full-length double-stranded DNA were designed to have a length of about 150 bases containing an overlapping region of 30 bases (SEQ ID NOS: 37 to 42). Six single-stranded oligo DNAs for synthesizing three short double-stranded DNA fragments corresponding to the fourth full-length double-stranded DNA were designed to have a length of about 150 bases containing an overlapping region of 30 bases (SEQ ID NOS: 43 to 48). The forward primer (SEQ ID NO: 49) and the reverse primer (SEQ ID NO: 50) for the overlap extension PCR reaction were also designed.

### (2) Two-Step Type Double-Stranded DNA Synthesis

The 24 single-stranded oligo DNAs required for synthesizing four double-stranded DNAs of interest having different-sequences were prepared at a concentration of 1 μM in sterile water or TE buffer (manufactured by Nacalai Tesque, Inc.).

For synthesizing short double-stranded DNA fragments required for synthesizing the first double-stranded DNA, three PCR reaction solutions A (1×Phusion HF Buffer, 0.2 mM dNTP, 0.016 U/μL Phusion High-Fidelity DNA polymerase) were prepared. To the first PCR reaction solution A, each 1 μL of single-stranded oligo DNA (SEQ ID NO: 25) and single-stranded oligo DNA (SEQ ID NO: 26) was added; to the second PCR reaction solution A, each 1 μL of single-stranded oligo DNA (SEQ ID NO: 27) and single-stranded oligo DNA (SEQ ID NO: 28) was added; and to the third PCR reaction solution A, each 1 μL of single-stranded oligo DNA (SEQ ID NO: 29) and single-stranded oligo DNA (SEQ ID NO: 30) was added. Each solution was prepared so that the total amount become 25 μL. PCR reaction solutions B (1×Phusion HF Buffer, 0.2 mM dNTP, 0.016 U/μL Phusion High-Fidelity DNA polymerase, 0.1 μM forward primer: SEQ ID NO: 49, 0.1 μM reverse primer: SEQ ID NO: 50) for synthesizing the first full-length double-stranded DNA was prepared. Each solution was prepared so that the total amount become 25 μL.

For synthesizing short double-stranded DNA fragments required for synthesizing the second double-stranded DNA, three PCR reaction solutions A (1×Phusion HF Buffer, 0.2 mM dNTP, 0.016 U/μL Phusion High-Fidelity DNA polymerase) were prepared. Thereafter, to the first PCR reaction solution A, each 1 μL of single-stranded oligo DNA (SEQ ID NO: 31) and single-stranded oligo DNA (SEQ ID NO: 32) was added; to the second PCR reaction solution A, each 1 μL of single-stranded oligo DNA (SEQ ID NO: 33) and single-stranded oligo DNA (SEQ ID NO: 34) was added; and to the third PCR reaction solution A, each 1 μL of single-stranded oligo DNA (SEQ ID NO: 35) and single-stranded oligo DNA (SEQ ID NO: 36) was added. Each solution was prepared so that the total amount become 25 PCR reaction solutions B (1×Phusion HF Buffer, 0.2 mM dNTP, 0.016 U/μL Phusion High-Fidelity DNA polymerase, 0.1 μM forward primer: SEQ ID NO: 49, 0.1 μM reverse primer: SEQ ID NO: 50) for synthesizing the second full-length double-stranded DNA was prepared. Each solution was prepared so that the total amount become 25 μL.

For synthesizing short double-stranded DNA fragments required for synthesizing the third double-stranded DNA, three PCR reaction solutions A (1×Phusion HF Buffer, 0.2 mM dNTP, 0.016 U/μL Phusion High-Fidelity DNA polymerase) were prepared. Thereafter, to the first PCR reaction solution A, each 1 μL of single-stranded oligo DNA (SEQ ID NO: 37) and single-stranded oligo DNA (SEQ ID NO: 38) was added; to the second PCR reaction solution A, each 1 μL of single-stranded oligo DNA (SEQ ID NO: 39) and single-stranded oligo DNA (SEQ ID NO: 40) was added; and to the third PCR reaction solution A, each 1 μL of single-stranded oligo DNA (SEQ ID NO: 41) and single-stranded oligo DNA (SEQ ID NO: 42) was added. Each solution was prepared so that the total amount become 25 μL. PCR reaction solutions B (1×Phusion Phusion HF Buffer, 0.2 mM dNTP, 0.016 U/μL Phusion High-Fidelity DNA polymerase, 0.1 μM forward primer: SEQ ID NO: 49, 0.1 μM reverse primer: SEQ ID NO: 50) for synthesizing the third full-length double-stranded DNA was prepared. Each solution was prepared so that the total amount become 25 μL.

For synthesizing short double-stranded DNA fragments required for synthesizing the fourth double-stranded DNA, three PCR reaction solutions A (1×Phusion HF Buffer, 0.2 mM dNTP, 0.016 U/μL Phusion High-Fidelity DNA polymerase) were prepared. Thereafter, to the first PCR reaction solution A, each 1 μL of single-stranded oligo DNA (SEQ ID NO: 43) and single-stranded oligo DNA (SEQ ID NO: 44) was added; to the second PCR reaction solution A, each 1 μL of single-stranded oligo DNA (SEQ ID NO: 45) and single-stranded oligo DNA (SEQ ID NO: 46) was added; and to the third PCR reaction solution A, each 1 μL of single-stranded oligo DNA (SEQ ID NO: 47) and single-stranded oligo DNA (SEQ ID NO: 48) was added. Each solution was prepared so that the total amount become 25 μL. PCR reaction solutions B (1×Phusion Phusion HF Buffer, 0.2 mM dNTP, 0.016 U/μL Phusion High-Fidelity DNA polymerase, 0.1 μM forward primer: SEQ ID NO: 49, 0.1 μM reverse primer: SEQ ID NO: 50) for synthesizing the fourth full-length double-stranded DNA was prepared. Each solution was prepared so that the total amount become 25 μL.

Using a thermal cycler manufactured by Takara Bio Inc., each of short double-stranded DNA fragments was synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., each 1 μL of the three PCR reaction solutions A of the short double-stranded DNA was added.

Further, using a thermal cycler manufactured by Takara Bio Inc., the three short double-stranded DNA fragments were connected to each other in multistage by overlap extension PCR reaction to synthesize a full-length double-stranded DNA. The temperature condition for the PCR reaction in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

Thereafter, a portion of the DNA amplification product of the double-stranded DNA synthesis was electrophoresed on a 1% agarose gel using an agarose gel electrophoresis device (Cosmo Bio Co., Ltd.). The results are shown in FIG. 6.

As shown in FIG. 6, the synthesis method of the present invention was able to simultaneously obtain the DNA amplification product of double-stranded DNA fragments of the four DNA sequences of interest. This result is achieved because, with the temperature holding period for each temperature gradient in the plurality of stages of the PCR reaction conditions, it is possible to complement the Tm values. That is, as compared to the conventional synthesis method, the synthesis methods of the present invention can simultaneously obtain a plurality of double-stranded DNA fragments of the DNA sequence of interest without optimizing sequence design.

The synthesis method of the present invention can obtain a double-stranded DNA fragment even when the DNA sequence of the fragment is a complex sequence such as a long repeat sequence, a sequence of contiguous identical nucleotides, or an AT-rich or GC-rich sequence, so that in the synthesis method of the present invention, the complexity of the DNA sequence of interest to be simultaneously synthesized is not particularly limited. According to the synthesis method of the present invention, it is possible to simultaneously synthesize a large number of double-stranded DNA fragments of several ten to several hundred units, thus the throughput property is significantly high. Further, it is also possible to automate the simultaneous synthesis of a plurality of double-stranded DNA fragments by applying a synthesis method of the present invention to a program used in a liquid dispensing robot.

The DNA amplification product of a plurality of double-stranded DNA fragments obtained by the synthesis method of the present invention can suppress the occurrence of non-specific DNA amplification products (DNA amplification products shorter in the length than DNA sequence of interest) significantly. Accordingly, the obtained DNA amplification product of double-stranded DNA fragments can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained.

In the synthesis method of a long chain DNA, a large number of double-stranded DNA fragments obtained by the synthesis method of double-stranded DNA fragments are used as the assembly materials to assemble the double-stranded DNA fragments, and thus even the lack of one double-stranded DNA fragment makes it impossible to synthesize the long chain DNA. In the conventional synthesis methods, it occurs inconvenience that it takes time and effort to synthesize a large number of double-stranded DNA fragments, and in some cases, the synthesis is difficult or impossible to synthesize depending on the complexity of the DNA sequence of interest to be synthesized. Thus, it has been difficult in the conventional methods to quickly obtain all of the double-stranded DNA fragments to be used as assembly materials. That is, the previous synthesis methods of double-stranded DNA fragments has failed to quickly supply the double-stranded DNA fragments to be used as assembly materials, which has been substantially a bottleneck for synthesizing a long-chain DNA. On the other hand, the synthesis method of the present invention can simultaneously synthesize a plurality of double-stranded DNA fragments regardless of the complexity of the DNA sequence of interest to be synthesized, so that it can quickly synthesize a large number of double-stranded DNA fragments which become assembly materials for synthesizing the long chain DNA. That is, the synthesis method of the present invention can supply quickly double-stranded DNA fragments required for synthesizing a long-chain DNA, and thus can significantly improve the throughput property of the synthesis method of long chain DNA.

### (3) Analysis of Chemically Synthesized Single-Stranded Oligo DNA Used in the Synthesis of Double-Stranded DNA

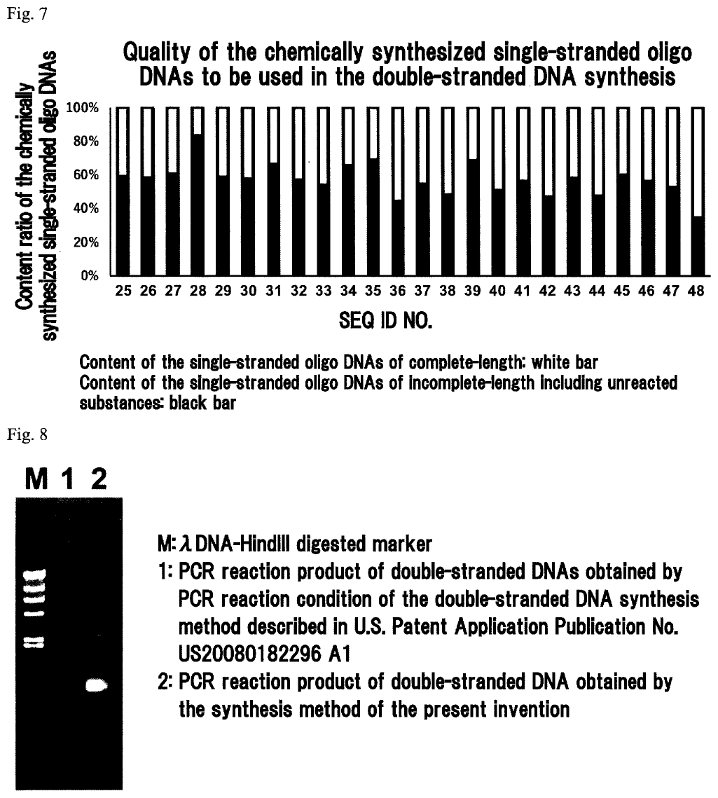

To analyze the chemically synthesized single-stranded oligo DNA to be used in the double-stranded DNA synthesis of the present invention, a single-stranded oligo DNA was gel-electrophoresed. Electrophoresis of single-stranded oligo DNA was performed using XCell SureLock Mini-Cell electrophoresis device (manufactured by Thermo Fisher Scientific Inc.) and 10% Novex TBE-Urea gel (manufactured by Thermo Fisher Scientific Inc.) in accordance with the attached instruction. Staining of single-stranded oligo DNA after electrophoresis was performed using a SYBR Green II nucleic acid fluorescence staining reagent manufactured by Takara Bio Inc. in accordance with the attached instruction. Gel analysis of single-stranded oligo DNA was performed using a gel imaging analyzer Gel Doc EZ system (manufactured by Bio-Rad Laboratories, Inc.) in accordance with the attached instruction. The results are shown in FIG. 7.

As shown in FIG. 7, with regard to the content of the single-stranded oligo DNA used in the synthesis method of the present invention, the content of single-stranded oligo DNA of complete-length was 60 to 15%, while the content of single-stranded oligo DNA of incomplete-length was 40 to 85%. The single-stranded oligo DNA of incomplete-length is reaction intermediate products such as unreacted substances generated in the course of chemical synthesis of single-stranded oligo DNA.

In the synthesis of double-stranded DNA fragments by the synthesis method of the present invention, it was used the single-stranded oligo DNAs containing 85% of single-stranded oligo DNA of incomplete-length (and 15% or more of single-stranded oligo DNA of complete-length). Thus, the synthesis method of the present invention was able to obtain a DNA amplification product of double-stranded DNA fragments of interest, even when the single-stranded oligo DNA of inferior quality containing around 85% of single-stranded oligo DNA of incomplete-length to the total of single-stranded oligo DNAs in the synthesis of the double-stranded DNA fragment was used. The obtained DNA amplification product of double-stranded DNA fragments can suppress the occurrence of non-specific DNA amplification products (DNA amplification products shorter in the length than DNA sequence of interest), so that it can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained.

### (Example 6) Cloning and Sequence Analysis of the Double-Stranded DNA Fragment Obtained by the Synthesis Method of the Present Invention

### (1) DNA Ligation and Transformation of the DNA Amplification Product of Double-Stranded DNA Fragments Obtained by the Synthesis Method of the Present Invention

To clone the DNA amplification product of double-stranded DNA synthesized in Example 5 described above, a dA protrusion was added to 3′-end of the DNA amplification product using 10× A-attachment Mix (manufactured by TOYOBO CO., LTD). Then, the DNA amplification product was purified using MinElute PCR Purification kit (manufactured by QIAGEN K.K.). Thereafter, the purified synthesized DNA and T-Vector pMD19 (Simple) (manufactured by Takara Bio Inc.) were mixed, and to the mixed DNA solution, DNA Ligation kit (manufactured by Takara Bio Inc.) was added to be the ratio of 1:1. The DNA ligation reaction solution was allowed to stand for 1 hour to overnight at 16° C. in a thermostat (manufactured by TAITEC CORPORATION). After the DNA ligation reaction, the transformation was performed using E. coli JM109 competent cells (manufactured by Takara Bio Inc.) in accordance with the attached instruction, and the resultant was spread on LB agar medium containing 100 μg/mL of carbenicillin (manufactured by Nacalai Tesque, Inc.), and cultured overnight at 37° C.

### (2) DNA Sequencing Analysis

A DNA extract were prepared from E. coli colonies using Cica geneus DNA extraction reagents (manufactured by Kanto Chemical Co., Inc.) in accordance with the attached instruction. Using the DNA extract as a template for PCR reaction, the DNA sequence of interest was amplified with M13 forward primer (SEQ ID NO: 51) and M13 reverse primer (SEQ ID NO: 52), using TaKaRa Ex-Taq Hot Start (manufactured by Takara Bio Inc.) in accordance with the attached instruction. The temperature condition for the PCR reaction in this case was 95° C. for 2 minutes and subsequent 30 cycles of the following temperature cycle: 95° C. for 20 seconds; 58° C. for 30 seconds; 72° C. for 60 seconds. A portion of the resulting PCR reaction product solution was electrophoresed on a 1% agarose gel (manufactured by Thermo Fisher Scientific Inc.). The remaining PCR reaction product solution was purified using MinElute PCR Purification kit (manufactured by QIAGEN K.K.) in accordance with the attached instruction.

The purified DNA product obtained above was subjected to sequencing reaction with M13 forward primer (SEQ ID NO: 51) and M13 reverse primer (SEQ ID NO: 52), using BigDye Terminator v3.1 Cycle Sequencing Kit (manufactured by Thermo Fisher Scientific Inc.) in accordance with the attached instruction. The reaction condition for this DNA sequencing was 95° C. for 2 minutes and subsequent 30 cycles of the following temperature cycle: 95° C. for 5 seconds; 50° C. for 10 seconds; 60° C. for 2 minutes and 30 seconds. The obtained DNA sequencing reaction product was purified using BigDye Terminator Purification kit (manufactured by Thermo Fisher Scientific Inc.) in accordance with the attached instruction. Then, the DNA sequence of interest was analyzed using Applied Biosystems 3500×L Genetic analyzer (manufactured by Thermo Fisher Scientific Inc.) in accordance with the attached instruction.

As the result of DNA sequence analysis, it has been found that it was possible to obtain cloned DNA of accurate sequence of the synthesized double-stranded DNA obtained by the synthesis method of the present invention.

### (Example 7) Comparison of the Double-Stranded DNA Fragments Obtained by the Conventional Synthesis Method to that Obtained by the Synthesis Method of the Present Invention-2

(Comparison of the PCR Reaction Condition of the Double-Stranded DNA Synthesis Method Described in U.S. Patent Application Publication No. US20080182296 A1 to the Double-Stranded DNA Synthesis Method of the Present Invention)

### (1) Sequence Design of Single-Stranded Oligo DNA

The sequence of full-length double-stranded DNA of interest was divided into three short double-stranded DNA fragments. These short double-stranded DNA fragments were designed so that the overlapping region of 30 base pairs was contained in the 5′- or 3′-end. Six single-stranded oligo DNAs for synthesizing these three short double-stranded DNA fragments were designed to have a length of about 150 bases containing an overlapping region of 30 bases (SEQ ID NOS: 53 to 58). The forward primer (SEQ ID NO: 59) and the reverse primer (SEQ ID NO: 60) for the overlap extension PCR reaction were also designed.

### (2) Two-Step Type Double-Stranded DNA Synthesis

The six single-stranded oligo DNAs required for synthesizing a double-stranded DNA were prepared at a concentration of 1 μM in sterile water or TE buffer (manufactured by Nacalai Tesque, Inc.). For synthesizing short double-stranded DNA fragments, three PCR reaction solutions A (1×Phusion HF Buffer, 0.2 mM dNTP, 0.016 U/μL Phusion High-Fidelity DNA polymerase) were prepared, and then, to the first PCR reaction solution A, each 1 μL of single-stranded oligo DNA (SEQ ID NO: 53) and single-stranded oligo DNA (SEQ ID NO: 54) was added; to the second PCR reaction solution A, each 1 μL of single-stranded oligo DNA (SEQ ID NO: 55) and single-stranded oligo DNA (SEQ ID NO: 56) was added; and to the third PCR reaction solution A, each 1 μL of single-stranded oligo DNA (SEQ ID NO: 57) and single-stranded oligo DNA (SEQ ID NO: 58) was added. Each solution was prepared so that the total amount become 25 μL. PCR reaction solutions B (1×Phusion HF Buffer, 0.2 mM dNTP, 0.016 U/μL Phusion High-Fidelity DNA polymerase, 0.1 μM forward primer: SEQ ID NO: 59, 0.1 μM reverse primer: SEQ ID NO: 60) for synthesizing the full-length double-stranded DNA was prepared. Each solution was prepared so that the total amount become 25 μL.

(i) Conventional Synthesis Method (the Method Described in U.S. patent application publication No. US20080182296 A1; Pcr-Directed Gene Synthesis from Large Number of Overlapping Oligodeoxyribonucleotides)

Using a thermal cycler manufactured by Takara Bio Inc., three short double-stranded DNA fragments were synthesized by primer extension PCR reaction. The temperature condition for the PCR reaction in this case was 95° C. for 4 minutes and subsequent 10 cycles of the following temperature cycle:

98° C. for 30 seconds; 50° C. for 30 seconds; 72° C. for 30 seconds.

Thereafter, the resultant was treated at 72° C. for 5 minutes, and then to the PCR reaction solution B maintaining at 72° C., each 1 μL of three PCR reaction solutions A of short double-stranded DNA was added.

Next, using a thermal cycler manufactured by Takara Bio Inc., the three short double-stranded DNA fragments were connected to each other in multistage by overlap extension PCR to synthesize a full-length double-stranded DNA. The temperature condition for the PCR reaction in this case was 95° C. for 4 minutes and subsequent 20 cycles of the following temperature cycle:

98° C. for 30 seconds; 50° C. for 30 seconds; 72° C. for 30 seconds.

Thereafter, the resultant was treated at 72° C. for 5 minutes.

### (ii) Synthesis Method of the Present Invention

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., each 1 μL of three PCR reaction solutions A of short double-stranded DNA was added.

Next, using a thermal cycler manufactured by Takara Bio Inc., the three short double- stranded DNA fragments were connected to each other in multistage by overlap extension PCR to synthesize a full-length double-stranded DNA. The temperature condition for the PCR reaction in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

Each portion of the DNA amplification products of double-stranded DNA fragments obtained by the PCR reaction condition of the conventional synthetic method (i) and the synthetic method of the present invention (ii) was electrophoresed on a 1% agarose gel using an agarose gel electrophoresis device (Cosmo Bio Co., Ltd.). The results are shown in FIG. 8.

As shown in FIG. 8, in the PCR reaction condition of the conventional synthesis method, the DNA amplification product of the double-stranded DNA fragment having a length of DNA sequence of interest could not be obtained. On the other hand, the synthesis method of the present invention was able to obtain a DNA amplification product of the double-stranded DNA fragment of the DNA sequence of interest. The obtained DNA amplification product of double-stranded DNA fragments can suppress the occurrence of non-specific DNA amplification products (DNA amplification products shorter in the length than DNA sequence of interest), so that it can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained.

Even when the DNA sequence of interest is difficult to synthesize in the PCR reaction condition in the conventional synthesis method (the methods described in U.S. Patent Application Publication No. US20080182296 A1), it is possible to obtain a double-stranded DNA fragment of the DNA sequence of interest by applying the synthesis method of the present invention. Accordingly, the synthesis method of the present invention, regardless of the complexity of the DNA sequence of interest, can synthesize a double-stranded DNA fragment of the DNA sequence of interest, thus the synthesis method of the present invention is superior to the conventional synthesis method.

### (Example 8) Comparison of the Double-Stranded DNA Fragments Obtained by the Conventional Synthesis Method to that Obtained by the Synthesis Method of the Present Invention-3

(Comparison with the Two-Step Type Double-Stranded DNA Synthesis Method in which all Single-Stranded Oligo DNA was Assembled in the First Step of the PCR Reaction Described in Stemmer, W. P., et al. Gene, 164, 49-53., 1995 to the Synthesis Method of Double-Stranded DNA of the Present Invention)

### (1) Sequence Design of Single-Stranded Oligo DNA

The sequence of a full-length double-stranded DNA of interest was divided into three short double-stranded DNA fragments. These short double-stranded DNA fragments were designed so that the overlapping region of 30 base pairs was contained in the 5′- or 3′-end. Six single-stranded oligo DNAs for synthesizing these three short double-stranded DNA fragments were designed to have a length of about 150 bases containing an overlapping region of 30 bases (SEQ ID NOS: 61 to 66). The forward primer (SEQ ID NO: 67) and the reverse primer (SEQ ID NO: 68) for the overlap extension PCR reaction were also designed.

### (2) Double-Stranded DNA Synthesis

The six single-stranded oligo DNAs required for synthesizing a double-stranded DNA were prepared at a concentration of 1 μM in sterile water or TE buffer (manufactured by Nacalai Tesque, Inc.).

(i) Conventional Synthesis Method (Two-Step Type Double-Stranded DNA Synthesis Method in which all Single-Stranded Oligo DNA was Assembled in the First Step of the PCR Reaction; Stemmer, W. P., et al. Gene, 164, 49-53., 1995)

To assemble all single-stranded oligo DNA in the first step, one PCR reaction solution A (1×Phusion HF Buffer, 0.2 mM dNTP, 0.016 U/μL Phusion High-Fidelity DNA polymerase) was prepared, and each 1 μL of the single-stranded oligo DNAs (SEQ ID NOS: 61 to 66) was added to the solution A. The solution was prepared so that the total amount become 25 μL. Then, PCR reaction solution B (1×Phusion HF Buffer, 0.2 mM dNTP, 0.016 U/μL Phusion High-Fidelity DNA polymerase, 0.1 μM forward primer: SEQ ID NO: 67, 0.1 μM reverse primer: SEQ ID NO: 68) for synthesizing the full-length double-stranded DNA was prepared. The solution was prepared so that the total amount become 25 μL.

Using a thermal cycler manufactured by Takara Bio Inc., all the single-stranded oligo DNA was assembled in the first step of the PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., 1 μL of the PCR reaction solution A of the assembled single-stranded oligo DNA was added.

Using a thermal cycler manufactured by Takara Bio Inc., the full-length double-stranded DNA was synthesized from the assembled single-stranded oligo DNA by the second step of the PCR reaction. The temperature condition for the PCR reaction in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

### (ii) Synthesis Method of the Present Invention

For synthesizing short double-stranded DNA fragments, three PCR reaction solutions A (1×Phusion HF Buffer, 0.2 mM dNTP, 0.016 U/μL Phusion High-Fidelity DNA polymerase) were prepared, and then, to the first PCR reaction solution A, each 1 μL of single-stranded oligo DNA (SEQ ID NO: 61) and single-stranded oligo DNA (SEQ ID NO: 62) was added; to the second PCR reaction solution A, each 1 μL of single-stranded oligo DNA (SEQ ID NO: 63) and single-stranded oligo DNA (SEQ ID NO: 64) was added; and to the third PCR reaction solution A, each 1 μL of single-stranded oligo DNA (SEQ ID NO: 65) and single-stranded oligo DNA (SEQ ID NO: 66) was added. Each solution was prepared so that the total amount become 25 μL. PCR reaction solutions B (1×Phusion HF Buffer, 0.2 mM dNTP, 0.016 U/μL Phusion High-Fidelity DNA polymerase, 0.1 μM forward primer: SEQ ID NO: 67, 0.1 μM reverse primer: SEQ ID NO: 68) for synthesizing the full-length double-stranded DNA was prepared. Each solution was prepared so that the total amount become 25 μL.

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, to the PCR reaction solution B maintaining at 72° C., each 1 μL of the three PCR reaction solutions A of short double-stranded DNA was added.

Using a thermal cycler manufactured by Takara Bio Inc., the three short double-stranded DNA fragments were connected to each other in multistage by overlap extension PCR reaction to synthesize a full-length double-stranded DNA. The temperature condition for the PCR reaction in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

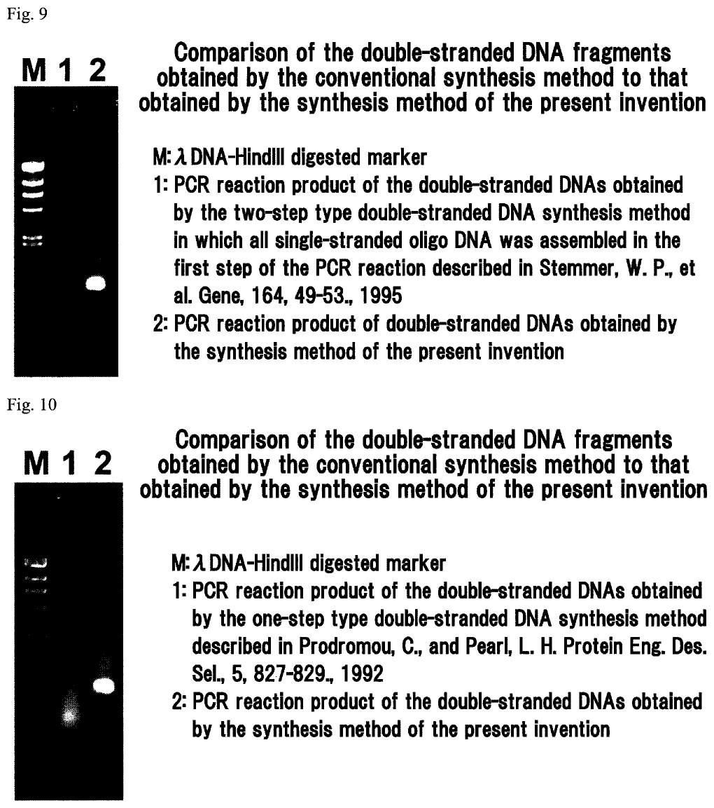

Each portion of the DNA amplification products of double-stranded DNA fragments obtained by the conventional synthetic methods (i) and the synthetic method of the present invention (ii) was electrophoresed on a 1% agarose gel using an agarose gel electrophoresis device (Cosmo Bio Co., Ltd.). The results are shown in FIG. 9.

As shown in FIG. 9, in the conventional synthetic methods, an DNA amplification product of the double-stranded DNA fragment having a length of DNA sequence of interest could not be obtained. On the other hand, the synthesis method of the present invention was able to obtain a DNA amplification product of the double-stranded DNA fragment of the DNA sequence of interest. From the results, it can be said that the synthesis method of the present invention was able to synthesize a double-stranded DNA of the DNA sequence of interest because it was possible to accurately synthesize the short double-stranded DNA by applying the primer extension PCR reaction to the first step of the synthesis method.

The obtained DNA amplification product of double-stranded DNA fragments can suppress the occurrence of non-specific DNA amplification products (DNA amplification products shorter in the length than DNA sequence of interest), so that it can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained.

Even when the DNA sequence of interest was difficult to synthesize in the conventional synthesis method, it is possible to obtain a double-stranded DNA fragment of the DNA sequence of interest by applying the synthesis method of the present invention. Accordingly, the synthesis method of the present invention, regardless of the complexity of the DNA sequence of interest, can synthesize a double-stranded DNA fragment of the DNA sequence of interest, thus the synthesis method of the present invention is superior to the conventional synthesis method.

### (Example 9) Comparison of the Double-Stranded DNA Fragments Obtained by the Conventional Synthesis Method to that Obtained by the Synthesis Method of the Present Invention-4

(Comparison of the One-Step Type Double-Stranded DNA Synthesis Method Described in Prodromou, C., and Pearl, L. H. Protein Eng. Des. Sel., 5, 827-829., 1992 to the Double-Stranded DNA Synthesis Method of the Present Invention)

### (1) Sequence Design of Single-Stranded Oligo DNA

The sequence of full-length double-stranded DNA of interest was divided into three short double-stranded DNA fragments. These short double-stranded DNA fragments were designed so that the overlapping region of 30 base pairs was contained in the 5′- or 3′-end. Six single-stranded oligo DNAs for synthesizing these three short double-stranded DNA fragments were designed to have a length of about 150 bases containing an overlapping region of 30 bases (SEQ ID NOS: 69 to 74). The forward primer (SEQ ID NO: 75) and the reverse primer SEQ ID NO: 76) for the overlap extension PCR reaction were also designed.

### (2) Double-Stranded DNA Synthesis

The six single-stranded oligo DNAs required for synthesizing a double-stranded DNA were prepared at a concentration of 1 μM in sterile water or TE buffer (manufactured by Nacalai Tesque, Inc.).

(i) Conventional Synthesis Method (One Stage Type Method for Synthesizing Double-Stranded DNA; Prodromou, C., and Pearl, L. H. Protein Eng. Des. Sel., 5, 827-829., 1992)

For synthesizing a double-stranded DNA from all single-stranded oligo DNAs, one PCR reaction solution A (1×Phusion HF Buffer, 0.2 mM dNTP, 0.016 U/μL Phusion High-Fidelity DNA polymerase, 0.1 μM forward primer: SEQ ID NO: 75, 0.1 μM reverse primer: SEQ ID NO: 76) was prepared, and each 1 μL of the single-stranded oligo DNAs (SEQ ID NOS: 69 to 74) was added to the solution A. The resultant solution was prepared so that the total amount become 25 μL.

Using a thermal cycler manufactured by Takara Bio Inc., single-stranded oligo DNAs were assembled by PCR reaction to synthesize the full-length double-stranded DNA. The temperature condition for the PCR reaction in this case was 98° C. for 2 minutes and subsequent 30 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

### (ii) Synthesis Method of the Present Invention

For synthesizing short double-stranded DNA fragments, three PCR reaction solutions A (1×Phusion HF Buffer, 0.2 mM dNTP, 0.016 U/μL Phusion High-Fidelity DNA polymerase) were prepared, and then, to the first PCR reaction solution A, each 1 μL of single-stranded oligo DNA (SEQ ID NO: 69) and single-stranded oligo DNA (SEQ ID NO: 70) was added; to the second PCR reaction solution A, each 1 μL of single-stranded oligo DNA (SEQ ID No. 71) and single-stranded oligo DNA (SEQ ID NO: 72) was added; and to the third PCR reaction solution A, each 1 μL of single-stranded oligo DNA (SEQ ID NO: 73) and single-stranded oligo DNA (SEQ ID NO: 74) was added. Each solution was prepared so that the total amount become 25 μL. PCR reaction solutions B (1×Phusion HF Buffer, 0.2 mM dNTP, 0.016 U/μL Phusion High-Fidelity DNA polymerase, 0.1 μM forward primer: SEQ ID NO: 75, 0.1 μM reverse primer: SEQ ID NO: 76) for synthesizing a full-length double-stranded DNA was prepared. Each solution was prepared so that the total amount become 25 μL.

Using a thermal cycler manufactured by Takara Bio Inc., three short double-stranded DNA fragments were synthesized by primer extension PCR reaction. The temperature condition for the PCR reaction in this case was 98° C. for 2 minutes and subsequent 10 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C., 50 seconds.

Thereafter, to the PCR reaction solution B maintaining at 72° C., each 1 μL of the three PCR reaction solutions A of the short double-stranded DNA was added.

Using a thermal cycler manufactured by Takara Bio Inc., the three short double-stranded DNA fragments were connected to each other in multistage by overlap extension PCR reaction to synthesize a full-length double-stranded DNA. The temperature condition for the PCR reaction in this case was 98° C. for 2 minutes and subsequent 20 cycles of the following temperature cycle:

98° C. for 30 seconds; 77.5° C. for 50 seconds; 75° C. for 50 seconds; 72.5° C. for 50 seconds; 70° C. for 50 seconds; 67.5° C. for 50 seconds; 65° C. for 50 seconds; 62.5° C. for 50 seconds; 72° C. for 50 seconds.

Thereafter, the resultant was treated at 72° C. for 3 minutes.

Each portion of DNA amplification products of double-stranded DNA fragments obtained by the conventional synthetic method (i) and the synthetic method of the present invention (ii) was electrophoresed on a 1% agarose gel using an agarose gel electrophoresis device (Cosmo Bio Co., Ltd.). The results are shown in FIG. 10.

As shown in FIG. 10, it could not be obtained in the conventional synthesis method a DNA amplification product of the double-stranded DNA fragment having a length of DNA sequence of interest. On the other hand, the synthesis method of the present invention was able to obtain a DNA amplification product of the double-stranded DNA fragment of the DNA sequence of interest. As a result, it can be said that the synthesis method of the present invention can synthesize the double-stranded DNA fragment of DNA sequence of interest by dividing the synthesis into two steps of primer extension PCR reaction and overlap extension PCR reaction.

The obtained DNA amplification product of double-stranded DNA fragments can suppress the occurrence of non-specific DNA amplification products (DNA amplification products shorter in the length than DNA sequence of interest), so that it can be applied to DNA cloning methods such as a TA cloning method and a blunt end cloning method, without going through a step of cutting out purification of a gel or other steps to take out only the double-stranded DNA fragment of interest, thus the cloned DNA containing the double-stranded DNA fragment of interest can be quickly obtained.

The synthesized DNA sequences in this Example were the DNA sequences containing long repeat sequence, a sequence of contiguous identical nucleotides, or an AT-rich region, those are generally said as complex sequences. Thus, it has been found that the two-step type double-stranded DNA synthesis method of the present invention can accurately synthesize double-stranded DNA of a DNA sequence of interest even when the DNA sequence is a complex sequence as described above.

Even when the DNA sequence of interest was difficult or impossible to synthesize in the conventional synthesis method, it is possible to obtain a double-stranded DNA fragment of the DNA sequence of interest by applying the synthesis method of the present invention. Accordingly, the synthesis method of the present invention, regardless of the complexity of the DNA sequence of interest, can synthesize a double-stranded DNA fragment of the DNA sequence of interest, thus the synthesis method of the present invention is superior to the conventional synthesis method.

### INDUSTRIAL APPLICABILITY

The method of the present invention, by having a plurality of stages of annealing temperature setting in a PCR cycle, can be applied to bonding of a plurality of DNAs having different Tm values, thus the method of the present invention can assemble a large number of single-stranded oligo DNAs containing homologous regions easily, accurately, efficiently, and quickly. Further, since the double-stranded DNA synthesis method of the present invention is applicable to the bonding of a plurality of DNAs having different Tm values, the method does not require labor to optimize the single-stranded oligo DNA sequence used as the material or need to delicately reset the temperature for each double-stranded DNA fragment of interest. Furthermore, the method of the present invention can synthesize double-stranded DNA fragment of interest easily, accurately, efficiently, and quickly, regardless of the sequence thereof, so that the method of the present invention can synthesis even a double-stranded DNA fragment having a conventional complex sequence (for example, a long repeat sequence, a sequence of contiguous identical nucleotides, or an AT-rich or GC-rich sequence) which has been hitherto difficult or impossible to synthesize.

1. A method for synthesizing a double-stranded DNA by connecting short double-stranded DNAs to each other by overlap extension PCR to obtain a double-stranded DNA fragment of interest, the method having a plurality of stages of annealing temperature setting in a PCR cycle of the overlap extension PCR.

2. The method for synthesizing the double-stranded DNA according to claim 1, wherein the annealing temperature setting is performed at 2 to 20 stages.

3. The method for synthesizing the double-stranded DNA according to claim 1 wherein an annealing temperature of the annealing temperature setting is 60 to 85° C.

4. The method for synthesizing the double-stranded DNA according to claim 1, wherein a temperature holding period in each stage of the annealing temperature setting is 10 seconds or more and 2 minutes or less.

5. The method for synthesizing the double-stranded DNA according to claim 1, wherein an overlapping region of DNA in the overlap extension PCR has 10 to 40 bases.

6. The method for synthesizing the double-stranded DNA according to claim 1, wherein 3 to 20 kinds of the short double-stranded DNAs are connected to each other in the overlap extension PCR.

7. The method for synthesizing the double-stranded DNA according to claim 1, wherein the short double-stranded DNAs used in the overlap extension PCR are synthesized by primer extension PCR.

8. The method for synthesizing the double-stranded DNA according to claim 7, wherein the method has a plurality of stages of annealing temperature setting in a PCR cycle of the primer extension PCR.

9. The method for synthesizing the double-stranded DNA according to claim 7, wherein a size of each of the short double-stranded DNAs synthesized by the primer extension PCR is 100 to 300 bases.

10. A method for synthesizing a double-stranded DNA, the method comprising a primer extension PCR step of synthesizing short double-stranded DNAs by primer extension PCR, and an overlap extension PCR step of connecting the double-stranded DNAs synthesized in the primer extension PCR step to each other by overlap extension PCR to synthesize a double-stranded DNA fragment of interest, and the method having a plurality of stages of annealing temperature setting in a PCR cycle of the overlap extension PCR.

11. The method for synthesizing the double-stranded DNA according to claim 2 wherein an annealing temperature of the annealing temperature setting is 60 to 85° C.

12. The method for synthesizing the double-stranded DNA according to claim 2, wherein a temperature holding period in each stage of the annealing temperature setting is 10 seconds or more and 2 minutes or less.

13. The method for synthesizing the double-stranded DNA according to claim 3, wherein a temperature holding period in each stage of the annealing temperature setting is 10 seconds or more and 2 minutes or less.

14. The method for synthesizing the double-stranded DNA according to claim 11, wherein a temperature holding period in each stage of the annealing temperature setting is 10 seconds or more and 2 minutes or less.

15. The method for synthesizing the double-stranded DNA according to claim 14, wherein an overlapping region of DNA in the overlap extension PCR has 10 to 40 bases.

16. The method for synthesizing the double-stranded DNA according to claim 15, wherein 3 to 20 kinds of the short double-stranded DNAs are connected to each other in the overlap extension PCR.

17. The method for synthesizing the double-stranded DNA according to claim 16, wherein the short double-stranded DNAs used in the overlap extension PCR are synthesized by primer extension PCR.


SEQUENCE LISTING (76 sequences)

SEQ ID NO: 1 - DNA, 154 bp, Artificial Sequence
  oligo DNA
tagaggcaagggttgtttttattgactagaagacctcgagtgtaacaagaacaaaatactttatggggtcgcacaagtcatgagagagatgtacgtagagatcgatcctgagacgcgaagtagccgtgccgccacttgggtgcacgaggaattc

SEQ ID NO: 2 - DNA, 154 bp, Artificial Sequence
  oligo DNA
ccttacgtttctctcttggaactagttgcacgtctagagcatcttcaattctttgttgaactactcgcaatgacgcctgacttaccggatgaatatccgaataaagcaatgagacgtgaggatggaattcctcgtgcacccaagtggcggcacg

SEQ ID NO: 3 - DNA, 154 bp, Artificial Sequence
  oligo DNA
tgcaactagttccaagagagaaacgtaagggttccggaaacgctgatgggtccaatgaagtgcaaatgagatgggattttgacgtatcttcatcgctatcttggaatatcccaggaacgtttaaagtggtcaactgtgtggggcccgttcaaga

SEQ ID NO: 4 - DNA, 154 bp, Artificial Sequence
  oligo DNA
ctaactagaacattttcaagtattgacagatatgaaaaaaaaaaaaaaaaaaaaaattctatataattacttacagtgcacataaatgtatgcttaaacgtccacccgtcccaatacctcccattcttgaacgggccccacacagttgaccact

SEQ ID NO: 5 - DNA, 154 bp, Artificial Sequence
  oligo DNA
tctgtcaatacttgaaaatgttctagttagagatttacgtatactgatcgcttgccttcgcaagggatggaaatcccttgaaaattaaaaaaatccccccctaagcagcgaaaattgtaagatccataatatttaagaaatgacgaagaacgta

SEQ ID NO: 6 - DNA, 124 bp, Artificial Sequence
  oligo DNA
tactggttggaagaaagacctctatgaagactgaggtggttgtggattatttgatgaatggcattctttgtttggaaaactgctgcgataagactacgttcttcgtcatttcttaaatattatg

SEQ ID NO: 7 - DNA, 28 bp, Artificial Sequence
  primer
tagaggcaagggttgtttttattgacta

SEQ ID NO: 8 - DNA, 25 bp, Artificial Sequence
  primer
tactggttggaagaaagacctctat

SEQ ID NO: 9 - DNA, 154 bp, Artificial Sequence
  oligo DNA
tagaggcaagggttgtttttattgactacacctgctatcgatgccgacttcggtgaaatttctgacccaagaaaattgcacagggaaatcaagttgttggttggtgttgttgaaaccggcttgtttattgataacgcttctaaggcttacttcg

SEQ ID NO: 10 - DNA, 154 bp, Artificial Sequence
  oligo DNA
agctttaataatacaatataaaaaatggagacactgttgtgaaaagtcaacctttactacggtttctgtctttgcctttgatctgcttacttctcagtaacttcaacagaaccatcagagttaccgaagtaagccttagaagcgttatcaataa

SEQ ID NO: 11 - DNA, 154 bp, Artificial Sequence
  oligo DNA
ctccattttttatattgtattattaaagctatttagttatttggatactgttttttttccagaagttttctttttagtaaagtacaatccagtaaaaatgaaggatgaacaatcggtgtatgcagattcaacaccaataaatgccacttggtgt

SEQ ID NO: 12 - DNA, 154 bp, Artificial Sequence
  oligo DNA
ctttcagaggagctgtgtattagcagatggtaagcgcacgttgaaaatgctgaatattattaatttagacataccttgatggctgaacagtagtcagaaaattaaaaaaaaaaaaaaagcgcttacaccaagtggcatttattggtgttgaatc

SEQ ID NO: 13 - DNA, 154 bp, Artificial Sequence
  oligo DNA
ccatctgctaatacacagctcctctgaaagtttcttggccgaatgtcataattttcgaaccaaatataaaaattcatacacattaaaacaagatgtgcttacaaagcggataagttccgcagcagccaaagctgcattgagacatttttcgcat

SEQ ID NO: 14 - DNA, 132 bp, Artificial Sequence
  oligo DNA
tactggttggaagaaagacctctatcacctgcaatgcaagtttgctgtattttagtcccgttggcgttttcggatgcttcattccccagagaagtgatgtgtatgcgaaaaatgtctcaatgcagctttggc

SEQ ID NO: 15 - DNA, 28 bp, Artificial Sequence
  primer
tagaggcaagggttgtttttattgacta

SEQ ID NO: 16 - DNA, 25 bp, Artificial Sequence
  primer
tactggttggaagaaagacctctat

SEQ ID NO: 17 - DNA, 187 bp, Artificial Sequence
  oligo DNA
tagcacctgcatgttcgtgcaattgttcgtagcgccatctttaaaggttgtcgtgcatttgttgtgatggaaggttatgaaggtctggttcgtggtggtccggaatatatcaaagaatttcattgggaagatgtgcgtggttggagcgcagaaggtggtacaaatattggcaccgcacgttgtatgg

SEQ ID NO: 18 - DNA, 187 bp, Artificial Sequence
  oligo DNA
ttggttttcagcagttcttcgatcaggctcggccattcgctacgaaacaggtctgcaccggtcaggctaccatcaccgccacaaacaatcagggcatcaacaccggcttcaatcagatgctgtgcacccagcagacgaccttcacgttttttgaattccatacaacgtgcggtgccaatatttgtac

SEQ ID NO: 19 - DNA, 187 bp, Artificial Sequence
  oligo DNA
gagcctgatcgaagaactgctgaaaaccaatcgtattagcaacgaacagtatgagcgtatgaaacatctgaatatttgtggcaccgttggcagcattgataatgatatgagcaccaccgatgcaaccattggtgcctatagcgcactggatcgtatttgtaaagccatcgattatgtggaagcaacc

SEQ ID NO: 20 - DNA, 187 bp, Artificial Sequence
  oligo DNA
caatatcacacatctgatcctgccattcactgctggttgccggtttttccggaataaagatataatctgcgctggttgcaatacctgccagcagtgccagccaaccacaattacgacccataacttcaaccacaaaggcacggctatggctatttgcggttgcttccacataatcgatggctttaca

SEQ ID NO: 21 - DNA, 187 bp, Artificial Sequence
  oligo DNA
gtgaatggcaggatcagatgtgtgatattgttagcaaacatcgtagccgtggtaaacgtaccaccattgttgttgttgccgaaggtgcaattgccgcagatctgaccccgattagcccgagtgatgttcataaagttctggttgatcgtctgggtttagatacccgtattaccacactgggtcatgt

SEQ ID NO: 22 - DNA, 181 bp, Artificial Sequence
  oligo DNA
tagcacctgcttgcgcacaattttgttttcattaacggcaatcagcggactcggtgtatccggtgtgctttccagaactgcattaactgcttccagaccttgcagggtcgccagaatacgatcatatgcaactgctgtaccaccacgctgaacatgacccagtgtggtaatacgggtatct

SEQ ID NO: 23 - DNA, 18 bp, Artificial Sequence
  primer
tagcacctgcatgttcgt

SEQ ID NO: 24 - DNA, 17 bp, Artificial Sequence
  primer
tagcacctgcttgcgca

SEQ ID NO: 25 - DNA, 154 bp, Artificial Sequence
  oligo DNA
tagaggcaagggttgtttttattgactacacctgcatgcattaggcctgtatggccgtccgggtgcatgcatgaacggtggtggcctactcaatggcgagcaaaacagtctcaagcggaagcagttggttcagacactgaataagcgtcacgga

SEQ ID NO: 26 - DNA, 154 bp, Artificial Sequence
  oligo DNA
agacgagatccttttcgacggcctgcttgacgggcgcgaagacgtactctagcgaaagcgggcgggctgccgcctcttctagcttgggtcccagggttagaggatctaccattgctagctcctctccgtgacgcttattcagtgtctgaaccaa

SEQ ID NO: 27 - DNA, 154 bp, Artificial Sequence
  oligo DNA
tcaagcaggccgtcgaaaaggatctcgtctctgttgggagcacctggtaagtaaggtgtagttttgcacccgtgtacataagcgtgaaatcaccacaaactgtgtgtatcaagtacatagtgacatttaaataatagcaagaacaacaataata

SEQ ID NO: 28 - DNA, 154 bp, Artificial Sequence
  oligo DNA
gccgaaaccgggcttttagcaccgttggaagcttgtatctccgcgtaagaccgccggagtatgcatcttatagcggtttttccagttcttttccactattacgtggtgcttccagtagcgctactattattgttgttcttgctattatttaaat

SEQ ID NO: 29 - DNA, 154 bp, Artificial Sequence
  oligo DNA
ttccaacggtgctaaaagcccggtttcggctcggccggaggaggaagagagacgaaaaaaaaaaaaatgactaaaaaaaaaatggaatattattaatgtgggatttttggctcaaggtgtggtggccccttttctaagggtggcgaattcttca

SEQ ID NO: 30 - DNA, 153 bp, Artificial Sequence
  oligo DNA
tactggttggaagaaagacctctatcacctgcgaggaagccactttaggtgttatgtgtcggtatatagatgaacccacagtttgcttatatatgggatagccttggcgagttttccgtacattgaagaattcgccacccttagaaaaggggc

SEQ ID NO: 31 - DNA, 154 bp, Artificial Sequence; identical to SEQ ID NO: 9
  oligo DNA

SEQ ID NO: 32 - DNA, 154 bp, Artificial Sequence; identical to SEQ ID NO: 10
  oligo DNA

SEQ ID NO: 33 - DNA, 154 bp, Artificial Sequence; identical to SEQ ID NO: 11
  oligo DNA

SEQ ID NO: 34 - DNA, 154 bp, Artificial Sequence; identical to SEQ ID NO: 12
  oligo DNA

SEQ ID NO: 35 - DNA, 154 bp, Artificial Sequence; identical to SEQ ID NO: 13
  oligo DNA

SEQ ID NO: 36 - DNA, 132 bp, Artificial Sequence; identical to SEQ ID NO: 14
  oligo DNA

SEQ ID NO: 37 - DNA, 154 bp, Artificial Sequence
  oligo DNA
tagaggcaagggttgtttttattgactacacctgcattacacgtcgtgaaaagctagaaaaggaattagacaccatcagaaacatggaaatctggaaggttggtaaggaagtcagaaagttgagaccagaaaaccaataaagaggaaaataata

SEQ ID NO: 38 - DNA, 154 bp, Artificial Sequence
  oligo DNA
cttcctagtgtacagatgtatgtcggatgaaaaaaaaaaaatcttaaatgtgaaattgggtcaattcaattgactatgacttgatgttgcaaaaattccaagagaaaaagtttccagcacttgatattattttcctctttattggttttctggt

SEQ ID NO: 39 - DNA, 154 bp, Artificial Sequence
  oligo DNA
tcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtg

SEQ ID NO: 40 - DNA, 154 bp, Artificial Sequence
  oligo DNA
catctttcaggccacgagggtagctcataacgccgcgctaatgccgctgcgtcacagcaaccagtagctcagccaaaaccgaaagagaaatcgtagctgtcccgatgaggacttatacacttgtcaccatctaaataaattatttattcgcgtt

SEQ ID NO: 41 - DNA, 154 bp, Artificial Sequence
  oligo DNA
ttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgc

SEQ ID NO: 42 - DNA, 140 bp, Artificial Sequence
  oligo DNA
tactggttggaagaaagacctctatcacctgcgtaaggaaattaaatacaggtgtacacatacacgcacacatatatatatatatatatatgtatatgtgtatataggaagcgcgcgcatgttagtatatacgattcgtt

SEQ ID NO: 43 - DNA, 154 bp, Artificial Sequence; identical to SEQ ID NO: 1
  oligo DNA

SEQ ID NO: 44 - DNA, 154 bp, Artificial Sequence; identical to SEQ ID NO: 2
  oligo DNA

SEQ ID NO: 45 - DNA, 154 bp, Artificial Sequence; identical to SEQ ID NO: 3
  oligo DNA

SEQ ID NO: 46 - DNA, 154 bp, Artificial Sequence; identical to SEQ ID NO: 4
  oligo DNA

SEQ ID NO: 47 - DNA, 154 bp, Artificial Sequence; identical to SEQ ID NO: 5
  oligo DNA

SEQ ID NO: 48 - DNA, 124 bp, Artificial Sequence; identical to SEQ ID NO: 6
  oligo DNA

SEQ ID NO: 49 - DNA, 28 bp, Artificial Sequence
  primer
tagaggcaagggttgtttttattgacta

SEQ ID NO: 50 - DNA, 25 bp, Artificial Sequence
  primer
tactggttggaagaaagacctctat

SEQ ID NO: 51 - DNA, 22 bp, Artificial Sequence
  primer
gttttcccagtcacgacgttgt

SEQ ID NO: 52 - DNA, 26 bp, Artificial Sequence
  primer
caggaaacagctatgaccatgattac

SEQ ID NO: 53 - DNA, 154 bp, Artificial Sequence; identical to SEQ ID NO: 1
  oligo DNA

SEQ ID NO: 54 - DNA, 154 bp, Artificial Sequence; identical to SEQ ID NO: 2
  oligo DNA

SEQ ID NO: 55 - DNA, 154 bp, Artificial Sequence; identical to SEQ ID NO: 3
  oligo DNA

SEQ ID NO: 56 - DNA, 154 bp, Artificial Sequence; identical to SEQ ID NO: 4
  oligo DNA

SEQ ID NO: 57 - DNA, 154 bp, Artificial Sequence; identical to SEQ ID NO: 5
  oligo DNA

SEQ ID NO: 58 - DNA, 124 bp, Artificial Sequence; identical to SEQ ID NO: 6
  oligo DNA

SEQ ID NO: 59 - DNA, 28 bp, Artificial Sequence
  primer
tagaggcaagggttgtttttattgacta

SEQ ID NO: 60 - DNA, 25 bp, Artificial Sequence
  primer
tactggttggaagaaagacctctat

SEQ ID NO: 61 - DNA, 154 bp, Artificial Sequence; identical to SEQ ID NO: 9
  oligo DNA

SEQ ID NO: 62 - DNA, 154 bp, Artificial Sequence; identical to SEQ ID NO: 10
  oligo DNA

SEQ ID NO: 63 - DNA, 154 bp, Artificial Sequence; identical to SEQ ID NO: 11
  oligo DNA

SEQ ID NO: 64 - DNA, 154 bp, Artificial Sequence; identical to SEQ ID NO: 12
  oligo DNA

SEQ ID NO: 65 - DNA, 154 bp, Artificial Sequence; identical to SEQ ID NO: 13
  oligo DNA

SEQ ID NO: 66 - DNA, 132 bp, Artificial Sequence; identical to SEQ ID NO: 14
  oligo DNA

SEQ ID NO: 67 - DNA, 28 bp, Artificial Sequence
  primer
tagaggcaagggttgtttttattgacta

SEQ ID NO: 68 - DNA, 25 bp, Artificial Sequence
  primer
tactggttggaagaaagacctctat

SEQ ID NO: 69 - DNA, 154 bp, Artificial Sequence; identical to SEQ ID NO: 9
  oligo DNA

SEQ ID NO: 70 - DNA, 154 bp, Artificial Sequence; identical to SEQ ID NO: 10
  oligo DNA

SEQ ID NO: 71 - DNA, 154 bp, Artificial Sequence; identical to SEQ ID NO: 11
  oligo DNA

SEQ ID NO: 72 - DNA, 154 bp, Artificial Sequence; identical to SEQ ID NO: 12
  oligo DNA

SEQ ID NO: 73 - DNA, 154 bp, Artificial Sequence; identical to SEQ ID NO: 13
  oligo DNA

SEQ ID NO: 74 - DNA, 132 bp, Artificial Sequence; identical to SEQ ID NO: 14
  oligo DNA

SEQ ID NO: 75 - DNA, 28 bp, Artificial Sequence
  primer
tagaggcaagggttgtttttattgacta

SEQ ID NO: 76 - DNA, 25 bp, Artificial Sequence
  primer
tactggttggaagaaagacctctat